(19) United States
(12) Patent Application Publication
(10) Pub. No.: US 2017/0016903 A1
(43) Pub. Date: Jan. 19, 2017

(54) GENES ENCODING SECRETED PROTEINS WHICH IDENTIFY CLINICALLY SIGNIFICANT PROSTATE CANCER

(71) Applicant: THE JOHNS HOPKINS UNIVERSITY, Baltimore, MD (US)

(72) Inventors: Ashley E. ROSS, Columbia, MD (US); Edward M. SCHAEFFER, Sparks, MD (US); Luigi MARCHIONNI, Baltimore, MD (US)

(21) Appl. No.: 15/121,783

(22) PCT Filed: Mar. 2, 2015
(86) PCT No.: PCT/US2015/018266

§ 371 Date: Aug. 26, 2016

Related U.S. Application Data
(60) Provisional application No. 61/945,959, filed on Feb. 28, 2014.

(51) Int. Cl. G01N 33/574

(52) U.S. Cl. CPC G01N 33/57434; G01N 2800/56; G01N 2800/60; G01N 2800/50; G01N 2800/52

(57) ABSTRACT

The present invention relates to the field of cancer. More specifically, the present invention provides compositions and methods for identification of individuals having clinically significant cancer. In one embodiment, a method for treating a subject having prostate cancer comprises the steps of (a) obtaining a biological sample from the subject; (b) performing an assay on the sample obtained from the subject to measure the levels of one or more protein biomarkers listed in Table 1; (c) comparing the measured levels of one or more protein biomarkers to one or more reference controls to identify the subject as having high grade prostate cancer, low grade prostate cancer or no prostate cancer; and (d) treating the subject with one or more treatment modalities appropriate for a subject having high grade prostate cancer or low grade prostate cancer.

### CROSS-REFERENCE TO RELATED APPLICATIONS

This application claims the benefit of U.S. Provisional Application No. 61/945,959, filed Feb. 28, 2014, which is incorporated herein by reference in its entirety.

### FIELD OF THE INVENTION

The present invention relates to the field of cancer. More specifically, the present invention provides compositions and methods for identification of individuals having clinically significant cancer.

### BACKGROUND OF THE INVENTION

Currently, triggers for prostate cancer biopsy include elevated PSAs, unfavorable PSA kinetics and digital rectal exam. Most recently developed blood tests include the use of [−2] proPSA, a key component of the Prostate Health Index, which is produced preferentially from the peripheral zone and shows modestly improved characteristics to predict the presence of prostate cancer on biopsy. Other recently commercialized tests include the use of urine markers for prostate cancer (PCA3). While PCA3 is specific for prostate cancer, it does not appear to distinguish indolent from lethal disease and thus will likely only contribute to an already large over diagnosis and treatment problem. Therefore, the next steps to reduce not only prostate cancer deaths but also the morbidity and costs associated with both over diagnosis and over treatment include the development of non-invasive methods which can identify clinically significant prostate cancer.

### SUMMARY OF THE INVENTION

The present invention is based, at least in part, on the discovery of a gene set whose differential expression is specific to clinically significant prostate cancer. Though prostate cancer represents a spectrum of disease pathologies with divergent outcomes clinically, a consensus exists that the presence of Gleason pattern 4 or 5 disease (Gleason score 7-10) signifies clinically significant cancer which in the majority of cases needs treatment. Because we wish to explore non-invasive methods for the detection of clinically significant prostate cancer, this list is based on genes coding for proteins that are thought to be secreted and thus might be more easily detected in the urine or blood. This is novel as currently there is no published assay which identifies clinically significant disease with adequate specificity in the blood or urine and there is not gene set based on secreted products that has been published for the discrimination of higher grade prostate cancer from low grade disease. As described more fully herein, the gene set comprises fibroblast growth factor 2 (FGF2), prostaglandin D2 synthase 21 kDa (PTGDS), glutathione peroxidase 3 (GPX3), pleiotrophin (PTN), serpin peptidase inhibitor, clade F member 1 (SERPINF1), angiopoietin-like 2 (ANGPTL2), sarcoglycan, beta (SGCB), latent transforming growth factor beta binding protein 4 (LTBP4), dystonin (DST), matrix metallopeptidase 2 (MMP2), serglycin (SRGN), dystrophin (DMD), fibulin 1 (FBLN1), endoplasmic reticulum aminopeptidase 1 (ERAP1), FXYD domain containing ion transport regulator 6 (FXYD6), lectin, galactoside-binding, soluble, 3 binding protein (LGALS3BP), angiogenin, ribonuclease, RNase A family, 5 (ANG), gelsolin (GSN), somatostatin (SST), dickkopf 3 homolog (DKK3).

Accordingly, in one aspect, the present invention provides methods for treating subjects that have prostate cancer. One aspect for treatment can include identifying whether the subject has prostate cancer. Another aspect includes identifying whether the subject has high grade or low grade prostate cancer, and then administering an appropriate treatment based on the identification. In one embodiment, a method for treating a subject having prostate cancer comprises the steps of (a) obtaining a biological sample from the subject; (b) performing an assay on the sample obtained from the subject to measure the levels of one or more protein biomarkers listed in Table 1; (c) comparing the measured levels of one or more protein biomarkers to one or more reference controls to identify the subject as having high grade prostate cancer, low grade prostate cancer or no prostate cancer; and (d) treating the subject with one or more treatment modalities appropriate for a subject having high grade prostate cancer or low grade prostate cancer. The treatment for high grade prostate cancer can include prostatectomy, radiation therapy, hormone therapy or chemotherapy.

In other embodiments, the gene set described herein (i.e., FGF2, PTGDS, GPX3, PTN, SERPINF1, ANGPTL2, SGCB, LTBP4, DST, MMP2, SRGN, DMD, FBLN1, ERAP1, FXYD6, LGALS3BP, ANG, GSN, SST, and DKK3) (one or more of the biomarkers or combinations thereof) can be used in methods to distinguish high grade vs. low grade prostate cancer (and direct appropriate treatment thereof), or to monitor the treatment thereof.

In particular embodiments, the present invention provides methods for treating subjects that have high grade prostate cancer. A method for treating a subject having high grade prostate cancer comprises the steps of (a) obtaining a biological sample from the subject; (b) performing an assay on the sample obtained from the subject to measure the levels of one or more protein biomarkers listed in Table 1; (c) identifying the subject as having high grade prostate cancer based on a comparison of the measured levels of one or more protein biomarkers to one or more reference controls; and (d) treating the subject with one or more treatment modalities appropriate for a subject having high grade prostate cancer.

In a specific embodiment, the assay of step (b) comprises contacting the biological sample with one or more capture agents that bind one or more protein biomarkers listed in Table 1 to form a capture agent:protein biomarker complex; and detecting/quantifying the capture agent:protein biomarker complexes. In another specific embodiment, the one or more capture agents are antibodies that specifically bind to one or more protein biomarkers listed in Table 1. In yet another embodiment, the assay of step (b) is an enzyme linked immunosorbent assay (ELISA).

In another specific embodiment, the one or more treatment modalities is prostatectomy, radiation therapy, hormone therapy or chemotherapy. In certain embodiments, the high grade prostate cancer comprises a Gleason score of 7 or higher. In particular embodiments, the one or more reference control levels comprise a high grade prostate cancer-positive reference control and/or a low grade prostate cancer-positive reference control.

In another aspect, the present invention provides methods for identifying subjects having high grade prostate cancer. In one embodiments, a method for identifying a subject as having high grade prostate cancer comprises the steps of (a) obtaining a biological sample from the subject; (b) performing an assay on the sample obtained from the subject to measure the levels of one or more of FGF2, PTGDS, GPX3, PTN, SERPINF1, ANGPTL2, SGCB, LTBP4, DST, MMP2, SRGN, DMD, FBLN1, ERAP1, FXYD6, LGALS3BP, ANG, GSN, SST, and DKK3; and (c) identifying the subject as having high grade prostate cancer based on a comparison of the measured levels of one or more protein biomarkers to one or more reference controls.

In a specific embodiment, the assay of step (b) comprises contacting the biological sample with one or more capture agents that bind one or more protein biomarkers listed in Table 1 to form a capture agent:protein biomarker complex; and detecting/quantifying the capture agent:protein biomarker complexes. In another specific embodiment, the one or more capture agents are antibodies that specifically bind to one or more protein biomarkers listed in Table 1. In yet another embodiment, the assay of step (b) is an enzyme linked immunosorbent assay (ELISA).

In certain embodiments, the one or more reference control levels comprise a high grade prostate cancer-positive reference control and/or a low grade prostate cancer-positive reference control. In the methods of the present invention, the biological sample can comprise blood, plasma, serum, urine or stool.

Accordingly, in one embodiment, the present invention provides a method for treating a patient having high grade prostate cancer comprising the steps of (a) obtaining a biological sample from the patient; (b) measuring the levels of one or more protein biomarkers listed in Table 1; and (c) classifying the patient as having high grade prostate cancer based on a comparison of the levels measured from the biological sample to a control or reference. In another embodiment, the method can further comprise (d) prescribing, recommending or treating the patient with one or more treatment modalities appropriate for a patient having high grade prostate cancer. In certain embodiments, the one or more treatment modalities is prostatectomy, radiation therapy, hormone therapy or chemotherapy.

In a specific embodiment, a method for treating high grade prostate cancer in a patient comprises the step of administering one or more treatment modalities appropriate for a patient having high grade prostate cancer to a patient classified as having high grade prostate cancer based on a comparison of protein expression data of a biomarker panel generated from a biological sample obtained from the patient to a control or reference, wherein the biomarker panel comprises one or more of FGF2, PTGDS, GPX3, PTN, SERPINF1, ANGPTL2, SGCB, LTBP4, DST, MMP2, SRGN, DMD, FBLN1, ERAP1, FXYD6, LGALS3BP, ANG, GSN, SST, and DKK3. The foregoing includes, for example, combinations of 2, 3, 4, 5, 6, 7, 8, 9, 10, 11, 12, 13, 14, 15, 16, 17, 18, 19, and 20 biomarkers.

In one embodiment, a method comprises the step of prescribing one or more treatment modalities appropriate for a patient having high grade prostate cancer to a patient classified as having high grade prostate cancer based on a comparison of protein expression data of a biomarker panel generated from a biological sample obtained from the patient to a control or reference, wherein the biomarker panel comprises one or more of FGF2, PTGDS, GPX3, PTN, SERPINF1, ANGPTL2, SGCB, LTBP4, DST, MMP2, SRGN, DMD, FBLN1, ERAP1, FXYD6, LGALS3BP, ANG, GSN, SST, and DKK3. In a further embodiment, a method comprises the steps of (a) prescribing one or more treatment modalities appropriate for a patient having high grade prostate cancer to a patient classified as having high grade prostate cancer based on a comparison of protein expression data of a biomarker panel generated from a biological sample obtained from the patient to a control or reference, wherein the biomarker panel comprises one or more of FGF2, PTGDS, GPX3, PTN, SERPINF1, ANGPTL2, SGCB, LTBP4, DST, MMP2, SRGN, DMD, FBLN1, ERAP1, FXYD6, LGALS3BP, ANG, GSN, SST, and DKK3; or (b) prescribing one or more treatment modalities appropriate for a patient having low grade prostate cancer to a patient classified as having low grade prostate cancer based on a comparison of protein expression data of a biomarker panel generated from a biological sample obtained from the patient to a control or reference, wherein the biomarker panel comprises one or more of FGF2, PTGDS, GPX3, PTN, SERPINF1, ANGPTL2, SGCB, LTBP4, DST, MMP2, SRGN, DMD, FBLN1, ERAP1, FXYD6, LGALS3BP, ANG, GSN, SST, and DKK3.

In yet another embodiment, a method comprises the steps of (a) ordering a diagnostic test that assays protein expression from a biological sample obtained from a patient and classifies the patient as having high grade prostate cancer based on a comparison of protein expression data of a biomarker panel generated from a biological sample obtained from the patient to a control or reference, wherein the biomarker panel comprises one or more of FGF2, PTGDS, GPX3, PTN, SERPINF1, ANGPTL2, SGCB, LTBP4, DST, MMP2, SRGN, DMD, FBLN1, ERAP1, FXYD6, LGALS3BP, ANG, GSN, SST, and DKK3; and (b) administering or prescribing one or more treatment modalities appropriate for a patient having high grade prostate cancer. In an alternative embodiment, (a) ordering a diagnostic test that assays protein expression from a biological sample obtained from a patient and classifies the patient as having low grade prostate cancer based on a comparison of protein expression data of a biomarker panel generated from a biological sample obtained from the patient to a control or reference, wherein the biomarker panel comprises one or more of FGF2, PTGDS, GPX3, PTN, SERPINF1, ANGPTL2, SGCB, LTBP4, DST, MMP2, SRGN, DMD, FBLN1, ERAP1, FXYD6, LGALS3BP, ANG, GSN, SST, and DKK3; and (b) administering or prescribing one or more treatment modalities appropriate for a patient having low grade prostate cancer. In a further embodiment, a method comprises the steps of (a) ordering a diagnostic test that assays protein expression from a biological sample obtained from a patient and classifies the patient as having high or low grade prostate cancer based on a comparison of protein expression data of a biomarker panel generated from a biological sample obtained from the patient to a control or reference, wherein the biomarker panel comprises one or more of FGF2, PTGDS, GPX3, PTN, SERPINF1, ANGPTL2, SGCB, LTBP4, DST, MMP2, SRGN, DMD, FBLN1, ERAP1, FXYD6, LGALS3BP, ANG, GSN, SST, and DKK3; and (b) administering or prescribing either (i) one or more treatment modalities appropriate for a patient having high grade prostate cancer to a patient classified high grade prostate cancer or (ii) one or more treatment modalities appropriate for a patient having low grade prostate cancer to a patient classified low grade prostate cancer.

### BRIEF DESCRIPTION OF THE FIGURES

FIG. 1: Laser capture microdissection of primary tumors with Gleason pattern 3, 4 or 5 disease as well as benign glands. RNA extracted was arrayed on the DASL platform as described in Ross et al. 2011. Differential gene expression between clinically significant (pattern 4 and above) and clinically insignificant cancer was determined and using Gene Ontology, differentially expressed genes coding for secreted products were selected. Genes with AUC to detect clinically significant prostate cancer were then determined by ROC analysis. A similar in silico analysis was performed on the Taylor et. al 2010, MSKCCC dataset. The top 20 concordantly differentially expressed genes with AUC greater than 0.7 for clinically significant disease are listed in Table 1.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

While prostate cancer screening has contributed to an overall decrease in prostate cancer specific mortality it has come at the price of over-diagnosis and overtreatment. Currently, due to limitations of PSA based screening, roughly half of diagnosed prostate cancers do not require treatment and have resulted in the exposure of men to invasive, and costly, diagnostic and treatment procedures. This invention describes a set of 20 genes whose differential expression correlates specifically with clinically significant prostate cancer (defined as Gleason sum 7-10 disease). The set comprises FGF2, PTGDS, GPX3, PTN, SERPINF1, ANGPTL2, SGCB, LTBP4, DST, MMP2, SRGN, DMD, FBLN1, ERAP1, FXYD6, LGALS3BP, ANG, GSN, SST, and DKK3. One or more of the foregoing biomarkers can be used in a method described herein.

### I. DEFINITIONS

As used herein, the term “antibody” is used in reference to any immunoglobulin molecule that reacts with a specific antigen. It is intended that the term encompass any immunoglobulin (e.g., IgG, IgM, IgA, IgE, IgD, etc.) obtained from any source (e.g., humans, rodents, non-human primates, caprines, bovines, equines, ovines, etc.). Specific types/examples of antibodies include polyclonal, monoclonal, humanized, chimeric, human, or otherwise-human-suitable antibodies. “Antibodies” also includes any fragment or derivative of any of the herein described antibodies.

As used herein, the term “antigen” is generally used in reference to any substance that is capable of reacting with an antibody. More specifically, as used herein, the term “antigen” refers to a biomarker described herein. An antigen can also refer to a synthetic peptide, polypeptide, protein or fragment of a polypeptide or protein, or other molecule which elicits an antibody response in a subject, or is recognized and bound by an antibody.

As used herein, the term “biomarker” refers to a molecule that is associated either quantitatively or qualitatively with a biological change. Examples of biomarkers include proteins, polypeptides, and fragments of a polypeptide or protein; and polynucleotides such as a gene product, RNA or RNA fragment. In certain embodiments, a “biomarker” means a molecule/compound that is differentially present (i.e., increased or decreased) in a biological sample as measured/compared against the same marker in another biological sample or control/reference. In other embodiments, a biomarker can be differentially present in a biological sample as measured/compared against the other markers in the same or another biological sample or control/reference. In further embodiments, one or more biomarkers can be differentially present in a biological sample as measured/compared against other markers in the same or another biological sample or control/reference and against the same markers in another biological sample or control/reference. In yet another embodiment, a biomarker can be differentially present in a biological sample from a subject or a group of subjects having a first phenotype (e.g., having a disease or condition) as compared to a biological sample from a subject or group of subjects having a second phenotype (e.g., not having the disease or condition or having a less severe version of the disease or condition).

In general, the one or more biomarkers can be generally present at a level that is increased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%, by at least 130%, by at least 140%, by at least 150%, or more; or is generally present at a level that is decreased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 100% (i.e., absent). A biomarker is preferably differentially present at a level that is statistically significant (e.g., a p-value less than 0.05 and/or a q-value of less than 0.10 as determined using, for example, either Welch's T-test or Wilcoxon's rank-sum Test). Biomarker levels can be used in conjunction with other parameters including, but not limited to, PSA, PCA3, and digital rectal exam, to assess a patient.

The “level” of one or more biomarkers means the absolute or relative amount or concentration of the biomarker in the sample.

“Non-biomarker compound” means a compound that is not differentially present in a biological sample from a subject or a group of subjects having a first phenotype (e.g., having a first disease) as compared to a biological sample from a subject or group of subjects having a second phenotype (e.g., not having the first disease). Such non-biomarker compounds may, however, be biomarkers in a biological sample from a subject or a group of subjects having a third phenotype (e.g., having a second disease) as compared to the first phenotype (e.g., having the first disease) or the second phenotype (e.g., not having the first disease).

“Prostate cancer” refers to a disease in which cancer develops in the prostate, a gland in the male reproductive system. “Low grade” or “lower grade” prostate cancer refers to non-metastatic prostate cancer, including malignant tumors with low potential for metastasis (i.e., prostate cancer that is considered to be “less aggressive”). Cancer tumors that are confined to the prostate (i.e., organ-confined, OC) are considered to be less aggressive prostate cancer. “High grade” or “higher grade” prostate cancer refers to prostate cancer that has metastasized in a subject, including malignant tumors with high potential for metastasis (prostate cancer that is considered to be “aggressive”). Cancer tumors that are not confined to the prostate (i.e., non-organ-confined, NOC) are considered to be aggressive prostate cancer. Tumors that are confined to the prostate (i.e., organ confined tumors) are considered to be less aggressive than tumors which are not confined to the prostate (i.e., non-organ confined tumors). “Aggressive” prostate cancer progresses, recurs and/or is the cause of death. Aggressive cancer may be characterized by one or more of the following: non-organ confined (NOC), association with extra capsular extensions (ECE), association with seminal vesicle invasion (SVI), association with lymph node invasion (LN), association with a Gleason Score major or Gleason Score minor of 4, and/or association with a Gleason Score Sum of 7 or higher. In contrast “less aggressive” cancer is confined to the prostate (organ confined, OC) and is not associated with extra capsular extensions (ECE), seminal vesicle invasion (SVI), lymph node invasion (LN), a Gleason Score major or Gleason Score minor of 4, or a Gleason Score Sum of 7 or higher. In certain embodiments, the terms high grade prostate cancer and aggressive prostate cancer can be used interchangeably. In particular embodiments, the terms low grade prostate cancer and less aggressive prostate cancer can be used interchangeably.

As used herein, the term “comparing” or “comparison” refers to making an assessment of how the proportion, level or cellular localization of one or more biomarkers in a sample from a patient relates to the proportion, level or cellular localization of the corresponding one or more biomarkers in a standard or control sample, or another sample (e.g., a pre-treatment sample). For example, “comparing” may refer to assessing whether the proportion, level, or cellular localization of one or more biomarkers in a sample from a patient is the same as, more or less than, or different from the proportion, level, or cellular localization of the corresponding one or more biomarkers in standard or control sample, or another sample. More specifically, the term may refer to assessing whether the proportion, level, or cellular localization of one or more biomarkers in a sample from a patient is the same as, more or less than, different from or otherwise corresponds (or not) to the proportion, level, or cellular localization of predefined biomarker levels/ratios that correspond to, for example, a patient having prostate cancer, not having prostate cancer, having high grade prostate cancer, having low grade prostate cancer, is responding to treatment for prostate cancer, is not responding to treatment for prostate cancer, is/is not likely to respond to a particular prostate cancer treatment, or having/not having another disease or condition. In a specific embodiment, the term “comparing” refers to assessing whether the level of one or more biomarkers of the present invention in a sample from a patient is the same as, more or less than, different from other otherwise correspond (or not) to levels/ratios of the same biomarkers in a control sample (e.g., predefined levels/ratios that correlate to high grade prostate cancer, low grade prostate cancer, uninfected individuals, standard prostate cancer levels/ratios, etc.).

In another embodiment, the term “comparing” refers to making an assessment of how the proportion, level or cellular localization of one or more biomarkers in a sample from a patient relates to the proportion, level or cellular localization of one or more biomarkers in the same sample. For example, a ratio of one biomarker to another (or more) from the same patient sample can be compared. Percentages or ratios of expression or levels of the biomarkers can be compared to other percentages or ratios in the same sample and/or to predefined reference or control percentages or ratios.

In embodiments in which the relationship of the biomarkers are described in terms of a ratio, the ratio can include 1-fold, 2-, 3-, 4-, 5-, 6-, 7-, 8-, 9-, 10-, 11-, 12-, 13-, 14-, 15-, 16-, 17-, 18-, 19-, 20-, 21-, 22-, 23-, 24-, 25-, 26-, 27-, 28-, 29-, 30-, 31-, 32-, 33-, 34-, 35-, 36-, 37-, 38-, 39-, 40-, 41-, 42-, 43-, 44-, 45-, 46-, 47-, 48-, 49-, 50-, 51-, 52-, 53-, 54-, 55-, 56-, 57-, 58-, 59-, 60-, 61-, 62-, 63-, 64-, 65-, 66-, 67-, 68-, 69-, 70-, 71-, 72-, 73-, 74-, 75-, 76-, 77-, 78-, 79-, 80-, 81-, 82-, 83-, 84-, 85-, 86-, 87-, 88-, 89-, 90-, 91-, 92-, 93-, 94-, 95-, 96-, 97-, 98-, 99-, 100-fold or more difference (higher or lower). Alternatively, the difference can include 0.9-fold, 0.8-fold, 0.7-fold, 0.7-fold, 0.6-fold, 0.5-fold, 0.4-fold, 0.3-fold, 0.2-fold, and 0.1-fold (higher or lower) depending on context. The foregoing can also be expressed in terms of a range (e.g., 1-5 fold/times higher or lower) or a threshold (e.g., at least 2-fold/times higher or lower).

The evaluation of the relationship between one or more biomarkers in a sample (e.g., one or more biomarkers compared to one or more other biomarkers (perhaps in combination with internal standards expression or levels (e.g., actin)) can also be expressed in terms of a percentage including, but not limited to, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 150%, 200% or more (higher or lower) difference. The foregoing can also be expressed in terms of a range (e.g., 50-100% higher or lower) or a threshold (e.g., at least 50% higher or lower)

As used herein, the terms “indicates” or “correlates” (or “indicating” or “correlating,” or “indication” or “correlation,” depending on the context) in reference to a parameter, e.g., a modulated proportion, level, or cellular localization in a sample from a patient, may mean that the patient has prostate cancer (high or low grade, for example). In specific embodiments, the parameter may comprise the level of one or more biomarkers of the present invention. A particular set or pattern of the amounts of one or more biomarkers may indicate that a patient has prostate cancer (i.e., correlates to a patient having prostate cancer (high or low grade, for example)). The terms can be used interchangeably with “identifying,” as in identifying based on a correlation, for example.

In other embodiments, a particular set or pattern of the amounts of one or more biomarkers may be correlated to a patient being unaffected (i.e., indicates a patient does not have prostate cancer). In certain embodiments, “indicating,” or “correlating,” as used according to the present invention, may be by any linear or non-linear method of quantifying the relationship between levels/ratios of biomarkers to a standard, control or comparative value for the assessment of the diagnosis, prediction of prostate cancer or prostate cancer progression, assessment of efficacy of clinical treatment, identification of a patient that may respond to a particular treatment regime or pharmaceutical agent, monitoring of the progress of treatment, and in the context of a screening assay, for the identification of an anti-prostate cancer therapeutic.

The terms “patient,” “individual,” or “subject” are used interchangeably herein, and refer to a mammal, particularly, a human. The patient may have a mild, intermediate or severe disease or condition. The patient may be treatment naïve, responding to any form of treatment, or refractory. The patient may be an individual in need of treatment or in need of diagnosis based on particular symptoms or family history. In some cases, the terms may refer to treatment in experimental animals, in veterinary application, and in the development of animal models for disease, including, but not limited to, rodents including mice, rats, and hamsters; and primates.

The terms “measuring” and “determining” are used interchangeably throughout, and refer to methods which include obtaining or providing a patient sample and/or detecting the level of a biomarker(s) in a sample. In one embodiment, the terms refer to obtaining or providing a patient sample and detecting the level of one or more biomarkers in the sample. In another embodiment, the terms “measuring” and “determining” mean detecting the level of one or more biomarkers in a patient sample. Measuring can be accomplished by methods known in the art and those further described herein. The term “measuring” is also used interchangeably throughout with the term “detecting.” In certain embodiments, the term is also used interchangeably with the term “quantitating.”

The terms “sample,” “patient sample,” “biological sample,” and the like, encompass a variety of sample types obtained from a patient, individual, or subject and can be used in a diagnostic or monitoring assay. The patient sample may be obtained from a healthy subject or a patient having symptoms associated with prostate cancer. Moreover, a sample obtained from a patient can be divided and only a portion may be used for diagnosis. Further, the sample, or a portion thereof, can be stored under conditions to maintain sample for later analysis. The definition specifically encompasses blood and other liquid samples of biological origin (including, but not limited to, peripheral blood, serum, plasma, cord blood, amniotic fluid, cerebrospinal fluid, urine, saliva, stool and synovial fluid), solid tissue samples such as a biopsy specimen or tissue cultures or cells derived therefrom and the progeny thereof. In certain embodiments, a sample comprises blood. In other embodiments, a sample comprises serum. In a specific embodiment, a sample comprises plasma. In another embodiment, a sample comprises urine. In yet another embodiment, a semen sample is used. In a further embodiment, a stool sample is used.

The definition of “sample” also includes samples that have been manipulated in any way after their procurement, such as by centrifugation, filtration, precipitation, dialysis, chromatography, treatment with reagents, washed, or enriched for certain cell populations. The terms further encompass a clinical sample, and also include cells in culture, cell supernatants, tissue samples, organs, and the like. Samples may also comprise fresh-frozen and/or formalin-fixed, paraffin-embedded tissue blocks, such as blocks prepared from clinical or pathological biopsies, prepared for pathological analysis or study by immunohistochemistry.

Various methodologies of the instant invention include a step that involves comparing a value, level, feature, characteristic, property, etc. to a “suitable control,” referred to interchangeably herein as an “appropriate control,” a “control sample,” a “reference” or simply a “control.” A “suitable control,” “appropriate control,” “control sample,” “reference” or a “control” is any control or standard familiar to one of ordinary skill in the art useful for comparison purposes. A “reference level” of a biomarker means a level of the biomarker that is indicative of a particular disease state, phenotype, or lack thereof, as well as combinations of disease states, phenotypes, or lack thereof. A “positive” reference level of a biomarker means a level that is indicative of a particular disease state or phenotype. A “negative” reference level of a biomarker means a level that is indicative of a lack of a particular disease state or phenotype. For example, a “prostate cancer-positive reference level” of a biomarker means a level of a biomarker that is indicative of a positive diagnosis of prostate cancer in a subject, and a “prostate cancer-negative reference level” of a biomarker means a level of a biomarker that is indicative of a negative diagnosis of prostate cancer in a subject. A “reference level” of a biomarker may be an absolute or relative amount or concentration of the biomarker, a presence or absence of the biomarker, a range of amount or concentration of the biomarker, a minimum and/or maximum amount or concentration of the biomarker, a mean amount or concentration of the biomarker, and/or a median amount or concentration of the biomarker; and, in addition, “reference levels” of combinations of biomarkers may also be ratios of absolute or relative amounts or concentrations of two or more biomarkers with respect to each other. Appropriate positive and negative reference levels of biomarkers for a particular disease state, phenotype, or lack thereof may be determined by measuring levels of desired biomarkers in one or more appropriate subjects, and such reference levels may be tailored to specific populations of subjects (e.g., a reference level may be age-matched so that comparisons may be made between biomarker levels in samples from subjects of a certain age and reference levels for a particular disease state, phenotype, or lack thereof in a certain age group). Such reference levels may also be tailored to specific techniques that are used to measure levels of biomarkers in biological samples (e.g., LC-MS, GC-MS, ELISA, PCR, etc.), where the levels of biomarkers may differ based on the specific technique that is used.

In one embodiment, a “suitable control” or “appropriate control” is a value, level, feature, characteristic, property, etc., determined in a cell, organ, or patient, e.g., a control or normal cell, organ, or patient, exhibiting, for example, normal traits. For example, the biomarkers of the present invention may be assayed for levels/ratios in a sample from an unaffected individual (UI) or a normal control individual (NC) (both terms are used interchangeably herein). In another embodiment, a “suitable control” or “appropriate control” is a value, level, feature, characteristic, property, ratio, etc. determined prior to performing a therapy (e.g., prostate cancer treatment) on a patient. In yet another embodiment, a transcription rate, mRNA level, translation rate, protein level/ratio, biological activity, cellular characteristic or property, genotype, phenotype, etc., can be determined prior to, during, or after administering a therapy into a cell, organ, or patient. In a further embodiment, a “suitable control” or “appropriate control” is a predefined value, level, feature, characteristic, property, ratio, etc. A “suitable control” can be a profile or pattern of levels/ratios of one or more biomarkers of the present invention that correlates to prostate cancer (high or low grade, for example), to which a patient sample can be compared. The patient sample can also be compared to a negative control, i.e., a profile that correlates to not having prostate cancer.

As used herein, the term “predetermined threshold value” of a biomarker refers to the level of the same biomarker in a corresponding control/normal sample or group of control/normal samples. Further, the term “altered level” of a biomarker in a sample refers to a level that is either below or above the predetermined threshold value for the same biomarker and thus encompasses either high (increased) or low (decreased) levels.

As used herein, the terms “binding agent specific for” or “binding agent that specifically binds” refers to an agent that binds to a biomarker and does not significantly bind to unrelated compounds. Examples of binding agents that can be effectively employed in the disclosed methods include, but are not limited to, lectins, proteins and antibodies, such as monoclonal or polyclonal antibodies, or antigen-binding fragments thereof, aptamers, etc. In certain embodiments, a binding agent binds a biomarker with an affinity constant of, for example, greater than or equal to about 1×10⁻⁶ M. A binding agent can also comprise a probe or primer that specifically hybridizes a biomarker nucleic acid.

The terms “specifically binds to,” “specific for,” and related grammatical variants refer to that binding which occurs between such paired species as enzyme/substrate, receptor/agonist, antibody/antigen, nucleic acid/complement and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody typically binds to a single epitope and to no other epitope within the family of proteins. In some embodiments, specific binding between an antigen and an antibody will have a binding affinity of at least 10⁻⁶ M. In other embodiments, the antigen and antibody will bind with affinities of at least 10⁻⁷ M, 10⁻⁸ M to 10⁻⁹ M, 10⁻¹⁰ M, 10⁻¹¹ M, or 10⁻¹² M.

### II. BINDING AGENTS

A binding agent is an agent that binds to a biomarker. The binding agent can be a capture agent, and the terms can be used interchangeably as the context indicates. For example, the capture agent can be a capture antibody that binds to an antigen on the biomarker. The capture agent can be coupled to a substrate and used to isolate the biomarker.

A binding agent can be DNA, RNA, monoclonal antibodies, polyclonal antibodies, Fabs, Fab′, single chain antibodies, synthetic antibodies, aptamers (DNA/RNA), peptoids, zDNA, peptide nucleic acids (PNAs), locked nucleic acids (LNAs), lectins, synthetic or naturally occurring chemical compounds (including but not limited to drugs, labeling reagents), dendrimers, or combinations thereof. For example, the binding agent can be a capture antibody.

In some instances, a single binding agent can be employed to isolate a biomarker. In other instances, a combination of different binding agents may be employed to isolate a biomarker. For example, at least 2, 3, 4, 5, 6, 7, 8, 9, 10, 11, 12, 13, 14, 15, 16, 17, 18, 19, 20, 25, 50, 75 or 100 different binding agents may be used to isolate a biomarker from a biological sample.

Different binding agents can also be used for multiplexing. For example, isolation of more than biomarker can be performed by isolating each biomarker with a different binding agent. Different binding agents can be bound to different particles, wherein the different particles are labeled. In another embodiment, an array comprising different binding agents can be used for multiplex analysis, wherein the different binding agents are differentially labeled or can be ascertained based on the location of the binding agent on the array. Multiplexing can be accomplished up to the resolution capability of the labels or detection method, such as described below.

The binding agent can be an antibody. For example, a biomarker may be isolated using one or more antibodies specific for one or more antigens present on the biomarker. The antibodies can be immunoglobulin molecules or immunologically active portions of immunoglobulin molecules, i.e., molecules that contain an antigen binding site that specifically binds an antigen and synthetic antibodies. The immunoglobulin molecules can be of any class (e.g., IgG, IgE, IgM, IgD or IgA) or subclass of immunoglobulin molecule. Antibodies include, but are not limited to, polyclonal, monoclonal, bispecific, synthetic, humanized and chimeric antibodies, single chain antibodies, Fab fragments and F(ab′)₂ fragments, Fv or Fv′ portions, fragments produced by a Fab expression library, anti-idiotypic (anti-Id) antibodies, or epitope-binding fragments of any of the above. An antibody, or generally any molecule, “binds specifically” to an antigen (or other molecule) if the antibody binds preferentially to the antigen, and, e.g., has less than about 30%, 20%, 10%, 5% or 1% cross-reactivity with another molecule.

The binding agent can also be a polypeptide or peptide. Polypeptide is used in its broadest sense and may include a sequence of subunit amino acids, amino acid analogs, or peptidomimetics. The subunits may be linked by peptide bonds. The polypeptides may be naturally occurring, processed forms of naturally occurring polypeptides (such as by enzymatic digestion), chemically synthesized or recombinantly expressed. The polypeptides for use in the methods of the present invention may be chemically synthesized using standard techniques. The polypeptides may comprise D-amino acids (which are resistant to L-amino acid-specific proteases), a combination of D- and L-amino acids, β amino acids, or various other designer or non-naturally occurring amino acids (e.g., β-methyl amino acids, Ca-methyl amino acids, and Nα-methyl amino acids, etc.) to convey special properties. Synthetic amino acids may include ornithine for lysine, and norleucine for leucine or isoleucine. In addition, the polypeptides can have peptidomimetic bonds, such as ester bonds, to prepare polypeptides with novel properties. For example, a polypeptide may be generated that incorporates a reduced peptide bond, i.e., R₁—CH₂—NH—R₂, where R₁ and R₂ are amino acid residues or sequences. A reduced peptide bond may be introduced as a dipeptide subunit. Such a polypeptide would be resistant to protease activity, and would possess an extended half-live in vivo. Polypeptides can also include peptoids (N-substituted glycines), in which the side chains are appended to nitrogen atoms along the molecule's backbone, rather than to the a-carbons, as in amino acids. Polypeptides and peptides are intended to be used interchangeably throughout this application, i.e., where the term peptide is used, it may also include polypeptides and where the term polypeptides is used, it may also include peptides.

A binding agent can also be linked directly or indirectly to a solid surface or substrate. A solid surface or substrate can be any physically separable solid to which a binding agent can be directly or indirectly attached including, but not limited to, surfaces provided by microarrays and wells, particles such as beads, columns, optical fibers, wipes, glass and modified or functionalized glass, quartz, mica, diazotized membranes (paper or nylon), polyformaldehyde, cellulose, cellulose acetate, paper, ceramics, metals, metalloids, semiconductive materials, quantum dots, coated beads or particles, other chromatographic materials, magnetic particles; plastics (including acrylics, polystyrene, copolymers of styrene or other materials, polypropylene, polyethylene, polybutylene, polyurethanes, TEFLON®, etc.), polysaccharides, nylon or nitrocellulose, resins, silica or silica-based materials including silicon and modified silicon, carbon, metals, inorganic glasses, plastics, ceramics, conducting polymers (including polymers such as polypyrole and polyindole); micro or nanostructured surfaces such as nucleic acid tiling arrays, nanotube, nanowire, or nanoparticulate decorated surfaces; or porous surfaces or gels such as methacrylates, acrylamides, sugar polymers, cellulose, silicates, or other fibrous or stranded polymers. In addition, as is known the art, the substrate may be coated using passive or chemically-derivatized coatings with any number of materials, including polymers, such as dextrans, acrylamides, gelatins or agarose. Such coatings can facilitate the use of the array with a biological sample.

For example, an antibody used to isolate a biomarker can be bound to a solid substrate such as a well, such as commercially available plates. Each well can be coated with the antibody. In some embodiments, the antibody used to isolate a biomarker can be bound to a solid substrate such as an array. The array can have a predetermined spatial arrangement of molecule interactions, binding islands, biomolecules, zones, domains or spatial arrangements of binding islands or binding agents deposited within discrete boundaries. Further, the term array may be used herein to refer to multiple arrays arranged on a surface, such as would be the case where a surface bore multiple copies of an array. Such surfaces bearing multiple arrays may also be referred to as multiple arrays or repeating arrays.

A binding agent can also be bound to particles such as beads or microspheres. For example, an antibody specific for a biomarker can be bound to a particle, and the antibody-bound particle is used to isolate biomarkers from a biological sample. In some embodiments, the microspheres may be magnetic or fluorescently labeled. In addition, a binding agent for isolating biomarkers can be a solid substrate itself. For example, latex beads, such as aldehyde/sulfate beads (Interfacial Dynamics, Portland, Oreg.) can be used.

A binding agent bound to a magnetic bead can also be used to isolate a biomarker. For example, a biological sample such as serum from a patient can be collected for prostate cancer screening. The sample can be incubated with an antibody to a biomarker coupled to magnetic microbeads. A low-density microcolumn can be placed in the magnetic field of a MACS Separator and the column is then washed with a buffer solution such as Tris-buffered saline. The magnetic immune complexes can then be applied to the column and unbound, non-specific material can be discarded. The selected biomarkers can be recovered by removing the column from the separator and placing it on a collection tube. A buffer can be added to the column and the magnetically labeled biomarkers can be released by applying the plunger supplied with the column. The isolated biomarkers can be diluted in IgG elution buffer and the complex can then be centrifuged to separate the microbeads from the biomarkers. The pelleted isolated cell-of-origin specific biomarkers can be resuspended in buffer such as phosphate-buffered saline and quantitated. Alternatively, due to the strong adhesion force between the antibody captured cell-of-origin specific biomarkers and the magnetic microbeads, a proteolytic enzyme such as trypsin can be used for the release of captured biomarkers without the need for centrifugation. The proteolytic enzyme can be incubated with the antibody captured cell-of-origin specific biomarkers for at least a time sufficient to release the biomarkers.

A binding agent, such as an antibody, for isolating a biomarker is preferably contacted with the biological sample comprising the biomarker of interest for at least a time sufficient for the binding agent to bind to the biomarker. For example, an antibody may be contacted with a biological sample for various intervals ranging from seconds days, including but not limited to, about 1, 2, 3, 4, 5, 6, 7, 8, 9, 10, 11, 12, 13, 14, 15, 16, 17, 18, 19. 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more minutes, 1, 2, 3, 4, 5, 6, 7, 8, 9, 10, 11, 12, 13, 14, 15, 16, 17, 18, 19. 20, 21, 22, 23, 24 or more hours, 1 day, 3 days, 7 days or 10 days.

A binding agent, such as an antibody specific to a biomarker described herein can be labeled with, but is not limited to, a magnetic label, a fluorescent moiety, an enzyme, a chemiluminescent probe, a metal particle, a non-metal colloidal particle, a polymeric dye particle, a pigment molecule, a pigment particle, an electrochemically active species, semiconductor nanocrystal or other nanoparticles including quantum dots or gold particles. The label can be, but not be limited to, fluorophores, quantum dots, or radioactive labels. For example, the label can be a radioisotope (radionuclides), such as ³H, ¹¹C, ¹⁴C, ¹⁸F, ³²P, ³⁵S, ⁶⁴Cu, ⁶⁸Ga, ⁸⁶Y, ⁹⁹Tc, ¹¹¹In, ¹²³I, ¹²⁴I, ¹²⁵I, ¹³¹I, ¹³³Xe, ³¹⁷⁷Lu, ²¹¹At, or ²¹³Bi. The label can be a fluorescent label, such as a rare earth chelate (europium chelate), fluorescein type, such as, but not limited to, FITC, 5-carboxyfluorescein, 6-carboxy fluorescein; a rhodamine type, such as, but not limited to, TAMRA; dansyl; Lissamine; cyanines; phycoerythrins; Texas Red; and analogs thereof.

A binding agent can be directly or indirectly labeled, e.g., the label can be attached to the antibody through biotin-streptavidin. Alternatively, an antibody is not labeled, but is later contacted with a second antibody that is labeled after the first antibody is bound to an antigen of interest.

For example, various enzyme-substrate labels are available or disclosed (see for example, U.S. Pat. No. 4,275,149). The enzyme generally catalyzes a chemical alteration of a chromogenic substrate that can be measured using various techniques. For example, the enzyme may catalyze a color change in a substrate, which can be measured spectrophotometrically. Alternatively, the enzyme may alter the fluorescence or chemiluminescence of the substrate. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRP), alkaline phosphatase (AP), β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Examples of enzyme-substrate combinations include, but are not limited to, horseradish peroxidase (HRP) with hydrogen peroxidase as a substrate, wherein the hydrogen peroxidase oxidizes a dye precursor (e.g., orthophenylene diamine (OPD) or 3,3′,5,5′-tetramethylbenzidine hydrochloride (TMB)); alkaline phosphatase (AP) with para-nitrophenyl phosphate as chromogenic substrate; and .beta.-D-galactosidase (β-D-Gal) with a chromogenic substrate (e.g., p-nitrophenyl-β-D-galactosidase) or fluorogenic substrate 4-methylumbelliferyl-β-D-galactosidase.

Depending on the method of isolation used, the binding agent may be linked to a solid surface or substrate, such as arrays, particles, wells and other substrates described above. Methods for direct chemical coupling of antibodies, to the cell surface are known in the art, and may include, for example, coupling using glutaraldehyde or maleimide activated antibodies. Methods for chemical coupling using multiple step procedures include biotinylation, coupling of trinitrophenol (TNP) or digoxigenin using for example succinimide esters of these compounds. Biotinylation can be accomplished by, for example, the use of D-biotinyl-N-hydroxysuccinimide. Succinimide groups react effectively with amino groups at pH values above 7, and preferentially between about pH 8.0 and about pH 8.5. Biotinylation can be accomplished by, for example, treating the cells with dithiothreitol followed by the addition of biotin maleimide.

### III. DETECTION OF PROSTATE CANCER BIOMARKERS

A. Detection by Immunoassay

In other embodiments, the biomarkers of the present invention can be detected and/or measured by immunoassay. Immunoassay requires biospecific binding reagents, such as antibodies, to capture the biomarkers. Many antibodies are available commercially. Antibodies also can be produced by methods well known in the art, e.g., by immunizing animals with the biomarkers. Biomarkers can be isolated from samples based on their binding characteristics. Alternatively, if the amino acid sequence of a polypeptide biomarker is known, the polypeptide can be synthesized and used to generate antibodies by methods well-known in the art.

The present invention contemplates traditional immunoassays including, for example, sandwich immunoassays including ELISA or fluorescence-based immunoassays, immunoblots, Western Blots (WB), as well as other enzyme immunoassays. Nephelometry is an assay performed in liquid phase, in which antibodies are in solution. Binding of the antigen to the antibody results in changes in absorbance, which is measured. In a SELDI-based immunoassay, a biospecific capture reagent for the biomarker is attached to the surface of an MS probe, such as a pre-activated protein chip array. The biomarker is then specifically captured on the biochip through this reagent, and the captured biomarker is detected by mass spectrometry.

Although antibodies are useful because of their extensive characterization, any other suitable agent (e.g., a peptide, an aptamer, or a small organic molecule) that specifically binds a biomarker of the present invention is optionally used in place of the antibody in the above described immunoassays. For example, an aptamer that specifically binds a biomarker and/or one or more of its breakdown products might be used. Aptamers are nucleic acid-based molecules that bind specific ligands. Methods for making aptamers with a particular binding specificity are known as detailed in U.S. Pat. No. 5,475,096; U.S. Pat. No. 5,670,637; U.S. Pat. No. 5,696,249; U.S. Pat. No. 5,270,163; U.S. Pat. No. 5,707,796; U.S. Pat. No. 5,595,877; U.S. Pat. No. 5,660,985; U.S. Pat. No. 5,567,588; U.S. Pat. No. 5,683,867; U.S. Pat. No. 5,637,459; and U.S. Pat. No. 6,011,020.

In specific embodiments, the assay performed on the biological sample can comprise contacting the biological sample with one or more binding/capture agents (e.g., antibodies, peptides, aptamer, etc., combinations thereof) to form a biomarker:binding agent complex. The complexes can then be detected and/or quantified. A subject can then be identified as having prostate cancer (high or low grade, for example) based on a comparison of the detected/quantified/measured levels of biomarkers to one or more reference controls as described herein.

In certain embodiments, the levels of the biomarkers employed herein are quantified by immunoassay, such as enzyme-linked immunoassay (ELISA) technology. In specific embodiments, the levels of expression of the biomarkers are determined by contacting the biological sample with antibodies, or antigen binding fragments thereof, that selectively bind to the biomarkers; and detecting binding of the antibodies, or antigen binding fragments thereof, to the biomarkers. In certain embodiments, the binding agents employed in the disclosed methods and compositions are labeled with a detectable moiety. For ease of reference, the term antibody is used in describing binding agents or capture molecules. However, it is understood that reference to an antibody in the context of describing an exemplary binding agent in the methods of the present invention also includes reference to other binding agents including, but not limited to lectins, peptides, aptamers and small organic molecules.

Furthermore, the level of a biomarker in a sample can be assayed by contacting the biological sample with an antibody, or antigen binding fragment thereof, that selectively binds to the target biomarker (referred to as a capture molecule or antibody or a binding agent), and detecting the binding of the antibody, or antigen-binding fragment thereof, to the biomarker. The detection can be performed using a second antibody to bind to the capture antibody complexed with its target biomarker. Kits for the detection of biomarkers as described herein can include pre-coated strip plates, biotinylated secondary antibody, standards, controls, buffers, streptavidin-horse radish peroxidise (HRP), tetramethyl benzidine (TMB), stop reagents, and detailed instructions for carrying out the tests including performing standards.

The present disclosure also provides methods in which the levels of the biomarkers in a biological sample are determined simultaneously. For example, in one embodiment, methods are provided that comprise: (a) contacting a biological sample obtained from the subject with a plurality of binding agents that selectively bind to a plurality of biomarkers disclosed herein for a period of time sufficient to form binding agent-biomarker complexes; and (b) detecting binding of the binding agents to the plurality of biomarkers, thereby determining the levels of the biomarkers in the biological sample. In a further embodiment, the method further comprises comparing the determined levels to a control or reference sample. In other embodiments, the method can further comprise generating a report summarizing the biomarker levels. In other embodiments, the method may further comprise recommending a particular treatment. For example, biomarker levels that are statistically significantly above or below control/reference levels indicates that the subject should be treated with one or more treatment modalities appropriate for a subject having high grade prostate cancer including prostatectomy, radiation therapy, hormone therapy or chemotherapy.

In a further aspect, the present disclosure provides compositions that can be employed in the disclosed methods. In certain embodiments, such compositions comprise a solid substrate and a plurality of binding agents immobilized on the substrate, wherein each of the binding agents is immobilized at a different, indexable, location on the substrate and the binding agents selectively bind to a plurality of biomarkers disclosed herein. In a specific embodiment, the locations are pre-determined. In other embodiments, kits are provided that comprise such compositions. In certain embodiments, the plurality of biomarkers includes one or more of the biomarkers described herein including FGF2, PTGDS, GPX3, PTN, SERPINF1, ANGPTL2, SGCB, LTBP4, DST, MMP2, SRGN, DMD, FBLN1, ERAP1, FXYD6, LGALS3BP, ANG, GSN, SST, and DKK3.

In a related aspect, methods for treating cancer in a patient/subject can comprise the steps of (a) contacting a biological sample obtained from the subject with a composition disclosed herein comprising binding agents for a period of time sufficient to form binding agent-biomarker complexes; (b) detecting binding of the binding agents to a plurality of biomarkers, thereby determining the levels of biomarkers in the biological sample; and (c) comparing the determined levels to a control or reference sample. In another embodiment, the method can further comprise the step of (d) treating the patient with one or more treatment modalities appropriate for a subject having high grade prostate cancer including prostatectomy, radiation therapy, hormone therapy or chemotherapy.

In specific embodiments, the assay performed on the biological sample can comprise contacting the biological sample with one or more capture agents (e.g., antibodies, lectins, peptides, aptamers, etc., combinations thereof) to form a biomarker:capture agent complex. The complexes can then be detected and/or quantified.

In one method, a first capture molecule or binding agent, such as an antibody that specifically binds the biomarker of interest, is immobilized on a suitable solid phase substrate or carrier. The test biological sample is then contacted with the capture antibody and incubated for a desired period of time. After washing to remove unbound material, a second, detection, antibody that binds to a different, non-overlapping, epitope on the biomarker is then used to detect binding of the biomarker to the capture antibody. The detection antibody is preferably conjugated, either directly or indirectly, to a detectable moiety. Examples of detectable moieties that can be employed in such methods include, but are not limited to, cheminescent and luminescent agents; fluorophores such as fluorescein, rhodamine and eosin; radioisotopes; colorimetric agents; and enzyme-substrate labels, such as biotin.

In another embodiment, the assay is a competitive binding assay, wherein labeled biomarker is used in place of the labeled detection antibody, and the labeled biomarker and any unlabeled biomarker present in the test sample compete for binding to the capture antibody. The amount of biomarker bound to the capture antibody can be determined based on the proportion of labeled biomarker detected.

Solid phase substrates, or carriers, that can be effectively employed in such assays are well known to those of skill in the art and include, for example, 96 well microtiter plates, glass, paper, chips and microporous membranes constructed, for example, of nitrocellulose, nylon, polyvinylidene difluoride, polyester, cellulose acetate, mixed cellulose esters and polycarbonate. Suitable microporous membranes include, for example, those described in US Patent Application Publication no. US 2010/0093557 A1. Methods for the automation of immunoassays are well known in the art and include, for example, those described in U.S. Pat. Nos. 5,885,530, 4,981,785, 6,159,750 and 5,358,691.

The presence of several different biomarkers in a test sample can be detected simultaneously using a multiplex assay, such as a multiplex ELISA. Multiplex assays offer the advantages of high throughput, a small volume of sample being required, and the ability to detect different proteins across a board dynamic range of concentrations.

In certain embodiments, such methods employ an array, wherein multiple binding agents (for example capture antibodies) specific for multiple biomarkers are immobilized on a substrate, such as a membrane, with each capture agent being positioned at a specific, pre-determined, location on the substrate. Methods for performing assays employing such arrays include those described, for example, in US Patent Application Publication nos. US2010/0093557A1 and US2010/0190656A1, the disclosures of which are hereby specifically incorporated by reference.

Multiplex arrays in several different formats based on the utilization of, for example, flow cytometry, chemiluminescence or electron-chemiluminesence technology, are well known in the art. Flow cytometric multiplex arrays, also known as bead-based multiplex arrays, include the Cytometric Bead Array (CBA) system from BD Biosciences (Bedford, Mass.) and multi-analyte profiling (xMAP®) technology from Luminex Corp. (Austin, Tex.), both of which employ bead sets which are distinguishable by flow cytometry. Each bead set is coated with a specific capture antibody. Fluorescence or streptavidin-labeled detection antibodies bind to specific capture antibody-biomarker complexes formed on the bead set. Multiple biomarkers can be recognized and measured by differences in the bead sets, with chromogenic or fluorogenic emissions being detected using flow cytometric analysis. In an alternative format, a multiplex ELISA from Quansys Biosciences (Logan, Utah) coats multiple specific capture antibodies at multiple spots (one antibody at one spot) in the same well on a 96-well microtiter plate. Chemiluminescence technology is then used to detect multiple biomarkers at the corresponding spots on the plate.

B. Detection by Polymerase Chain Reaction

In certain embodiments, the biomarkers of the present invention can be detected/measure/quantitated by polymerase chain reaction (PCR). In certain embodiments, the present invention contemplates quantitation of one or more biomarkers described herein including FGF2, PTGDS, GPX3, PTN, SERPINF1, ANGPTL2, SGCB, LTBP4, DST, MMP2, SRGN, DMD, FBLN1, ERAP1, FXYD6, LGALS3BP, ANG, GSN, SST, and DKK3. The one or more biomarkers can be quantitated and the expression can be compared to reference levels. Overexpression relative to the reference is indicative of cancer. PCR can include quantitative type PCR, such as quantitative, real-time PCR (both singleplex and multiplex). In a specific embodiments, the quantitation steps are carried using quantitative, real-time PCR. One of ordinary skill in the art can design primers that specifically bind and amplify one or more biomarkers described herein using the publicly available sequences thereof.

In more particular embodiments, an assay performed on a biological sample obtained from a subject may comprise extracting nucleic acids from the biological sample. The assay can further comprise contacting nucleic acids with one or more primers that specifically bind one or more biomarker listed in Table 1 to form a primer:biomarker complex. The assay can further comprise the step of amplifying the primer:biomarker compexes. The amplified complexes can then be detected/quantified to determine a level of expression of the one or more biomarkers. A subject can then be identified as having high grade prostate cancer (or low grade or no prostate cancer) based on a comparison of the measure/quantified/determined levels of one or more biomarkers in Table 1 to one or more reference controls as described herein. The subject can then be treated appropriately, based on the grade of prostate cancer. The assay can be performed on mRNA extracted from the biological sample.

Many methods of measuring the levels or amounts of biomarker nucleic acid expression are contemplated. Any reliable, sensitive, and specific method can be used. In particular embodiments, biomarker nucleic acid is amplified prior to measurement. In other embodiments, the level of biomarker nucleic acid is measured during the amplification process. In still other methods, the target nucleic acid is not amplified prior to measurement.

1. Amplification Reactions

Many methods exist for amplifying nucleic acid sequences. Suitable nucleic acid polymerization and amplification techniques include reverse transcription (RT), polymerase chain reaction (PCR), real-time PCR (quantitative PCR (q-PCR)), nucleic acid sequence-base amplification (NASBA), ligase chain reaction, multiplex ligatable probe amplification, invader technology (Third Wave), rolling circle amplification, in vitro transcription (IVT), strand displacement amplification, transcription-mediated amplification (TMA), RNA (Eberwine) amplification, and other methods that are known to persons skilled in the art. In certain embodiments, more than one amplification method is used, such as reverse transcription followed by real time quantitative PCR (qRT-PCR). See, e.g., Chen et al., 33(20) NUCL. ACIDS RES. e179 (2005).

A typical PCR reaction comprises multiple amplification steps or cycles that selectively amplify target nucleic acid species including a denaturing step in which a target nucleic acid is denatured; an annealing step in which a set of PCR primers (forward and reverse primers) anneal to complementary DNA strands; and an extension step in which a thermostable DNA polymerase extends the primers. By repeating these steps multiple times, a DNA fragment is amplified to produce an amplicon, corresponding to the target DNA sequence. Typical PCR reactions include about 20 or more cycles of denaturation, annealing, and extension. In many cases, the annealing and extension steps can be performed concurrently, in which case the cycle contains only two steps. Because mature mRNA are single-stranded, a reverse transcription reaction (which produces a complementary cDNA sequence) may be performed prior to PCR reactions. Reverse transcription reactions include the use of, e.g., a RNA-based DNA polymerase (reverse transcriptase) and a primer.

In PCR and q-PCR methods, for example, a set of primers is used for each target sequence. In certain embodiments, the lengths of the primers depends on many factors, including, but not limited to, the desired hybridization temperature between the primers, the target nucleic acid sequence, and the complexity of the different target nucleic acid sequences to be amplified. In certain embodiments, a primer is about 15 to about 35 nucleotides in length. In other embodiments, a primer is equal to or fewer than about 15, fewer than about 20, fewer than about 25, fewer than about 30, or fewer than about 35 nucleotides in length. In additional embodiments, a primer is at least about 35 nucleotides in length.

In a further embodiment, a forward primer can comprise at least one sequence that anneals to biomarker nucleic acid sequence and alternatively can comprise an additional 5′ non-complementary region. In another embodiment, a reverse primer can be designed to anneal to the complement of a reverse transcribed mRNA. The reverse primer may be independent of the biomarker nucleic acid sequence, and multiple biomarker nucleic acid sequences may be amplified using the same reverse primer. Alternatively, a reverse primer may be specific for a biomarker nucleic acid.

In some embodiments, two or more biomarker nucleic acid sequences are amplified in a single reaction volume. One aspect includes multiplex q-PCR, such as qRT-PCR, which enables simultaneous amplification and quantification of at least two biomarker nucleic acid sequences of interest in one reaction volume by using more than one pair of primers and/or more than one probe. The primer pairs comprise at least one amplification primer that uniquely binds each mRNA, and the probes are labeled such that they are distinguishable from one another, thus allowing simultaneous quantification of multiple biomarker nucleic acid sequences. Multiplex qRT-PCR has research and diagnostic uses including, but not limited, to detection of biomarker nucleic acid sequences for diagnostic, prognostic, and therapeutic applications.

The qRT-PCR reaction may further be combined with the reverse transcription reaction by including both a reverse transcriptase and a DNA-based thermostable DNA polymerase. When two polymerases are used, a “hot start” approach may be used to maximize assay performance. See U.S. Pat. No. 5,985,619 and U.S. Pat. No. 5,411,876. For example, the components for a reverse transcriptase reaction and a PCR reaction may be sequestered using one or more thermoactivation methods or chemical alteration to improve polymerization efficiency. See U.S. Pat. No. 6,403,341; U.S. Pat. No. 5,550,044; and U.S. Pat. No. 5,413,924.

2. Detection of Target Biomarker Nucleic Acids

In certain embodiments, labels, dyes, or labeled probes and/or primers are used to detect amplified or unamplified biomarker nucleic acid sequence (mRNA/cDNA). One of ordinary skill in the art will recognize which detection methods are appropriate based on the sensitivity of the detection method and the abundance of the target. Depending on the sensitivity of the detection method and the abundance of the target, amplification may or may not be required prior to detection. One skilled in the art will recognize the detection methods where biomarker nucleic acid sequence amplification is preferred.

A probe or primer may include Watson-Crick bases or modified bases. Modified bases include, but are not limited to, the AEGIS bases (from EraGen Biosciences, Inc. (Madison, Wis.)), which have been described, e.g., in U.S. Pat. No. 6,001,983; U.S. Pat. No. 5,965,364; and U.S. Pat. No. 5,432,272. In certain aspects, bases are joined by a natural phosphodiester bond or a different chemical linkage. Different chemical linkages include, but are not limited to, a peptide bond or a Locked Nucleic Acid (LNA) linkage, which is described, e.g., in U.S. Pat. No. 7,060,809.

In a further aspect, oligonucleotide probes or primers present in an amplification reaction are suitable for monitoring the amount of amplification product produced as a function of time. In certain aspects, probes having different single stranded versus double stranded character are used to detect the nucleic acid. Probes include, but are not limited to, the 5′-exonuclease assay (e.g., TaqMan®) probes (see U.S. Pat. No. 5,538,848), stem-loop molecular beacons (see, e.g., U.S. Pat. No. 6,103,476 and U.S. Pat. No. 5,925,517), stemless or linear beacons (see, e.g., WO 9921881, U.S. Pat. No. 6,649,349 and U.S. Pat. No. 6,485,901), peptide nucleic acid (PNA) Molecular Beacons (see, e.g., U.S. Pat. No. 6,593,091 and U.S. Pat. No. 6,355,421), linear PNA beacons (see, e.g., U.S. Pat. No. 6,329,144), non-FRET probes (see, e.g., U.S. Pat. No. 6,150,097), Sunrise®/Amplifluor® probes (see, e.g., U.S. Pat. No. 6,548,250), stem-loop and duplex Scorpion™ probes (see, e.g., U.S. Pat. No. 6,589,743), bulge loop probes (see, e.g., U.S. Pat. No. 6,590,091), pseudo knot probes (see, e.g., U.S. Pat. No. 6,548,250), cyclicons (see, e.g., U.S. Pat. No. 6,383,752), MGB Eclipse® probe (Sigma-Aldrich Corp. (St. Louis, Mo.)), hairpin probes (see, e.g., U.S. Pat. No. 6,596,490), PNA light-up probes, antiprimer quench probes (Li et al., 53 CLIN. CHEM. 624-33 (2006)), self-assembled nanoparticle probes, and ferrocene-modified probes described, for example, in U.S. Pat. No. 6,485,901.

In certain embodiments, one or more of the primers in an amplification reaction can include a label. In yet further embodiments, different probes or primers comprise detectable labels that are distinguishable from one another. In some embodiments a nucleic acid, such as the probe or primer, may be labeled with two or more distinguishable labels.

In some aspects, a label is attached to one or more probes and has one or more of the following properties: (i) provides a detectable signal; (ii) interacts with a second label to modify the detectable signal provided by the second label, e.g., FRET (Fluorescent Resonance Energy Transfer); (iii) stabilizes hybridization, e.g., duplex formation; and (iv) provides a member of a binding complex or affinity set, e.g., affinity, antibody-antigen, ionic complexes, hapten-ligand (e.g., biotin-avidin). In still other aspects, use of labels can be accomplished using any one of a large number of known techniques employing known labels, linkages, linking groups, reagents, reaction conditions, and analysis and purification methods.

Biomarker nucleic acid sequences can be detected by direct or indirect methods. In a direct detection method, one or more biomarker nucleic acid sequences are detected by a detectable label that is linked to a nucleic acid molecule. In such methods, the biomarker nucleic acid sequences may be labeled prior to binding to the probe. Therefore, binding is detected by screening for the labeled biomarker nucleic acid sequence that is bound to the probe. The probe is optionally linked to a bead in the reaction volume.

In certain embodiments, nucleic acids are detected by direct binding with a labeled probe, and the probe is subsequently detected. In one embodiment of the invention, the nucleic acids, such as amplified mRNA/cDNA, are detected using xMAP Microspheres (Luminex Corp. (Austin, Tex.)) conjugated with probes to capture the desired nucleic acids. Some methods may involve detection with polynucleotide probes modified, for example, with fluorescent labels or branched DNA (bDNA) detection.

In other embodiments, nucleic acids are detected by indirect detection methods. For example, a biotinylated probe may be combined with a stretavidin-conjugated dye to detect the bound nucleic acid. The streptavidin molecule binds a biotin label on amplified nucleic acid, and the bound nucleic acid is detected by detecting the dye molecule attached to the streptavidin molecule. In one embodiment, the streptavidin-conjugated dye molecule comprises Phycolink® Streptavidin R-Phycoerythrin (ProZyme, Inc. (Heward, Calif.)). Other conjugated dye molecules are known to persons skilled in the art.

Labels include, but are not limited to, light-emitting, light-scattering, and light-absorbing compounds which generate or quench a detectable fluorescent, chemiluminescent, or bioluminescent signal. See, e.g., Garman A., Non-Radioactive Labeling, Academic Press (1997) and Kricka, L., Nonisotopic DNA Probe Techniques, Academic Press, San Diego (1992). Fluorescent reporter dyes useful as labels include, but are not limited to, fluoresceins (see, e.g., U.S. Pat. No. 6,020,481; U.S. Pat. No. 6,008,379; and U.S. Pat. No. 5,188,934), rhodamines (see, e.g., U.S. Pat. No. 6,191,278; U.S. Pat. No. 6,051,719; U.S. Pat. No. 5,936,087; U.S. Pat. No. 5,847,162; and U.S. Pat. No. 5,366,860), benzophenoxazines (see, e.g., U.S. Pat. No. 6,140,500), energy-transfer fluorescent dyes, comprising pairs of donors and acceptors (see, e.g., U.S. Pat. No. 5,945,526; U.S. Pat. Nos. 5,863,727; and 5,800,996; and), and cyanines (see, e.g., WO 9745539), lissamine, phycoerythrin, Cy2, Cy3, Cy3.5, Cy5, Cy5.5, Cy7, FluorX (Amersham Biosciences, Inc. (Piscataway, N.J.)), Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5, 6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, Tetramethylrhodamine, and/or Texas Red, as well as any other fluorescent moiety capable of generating a detectable signal. Examples of fluorescein dyes include, but are not limited to, 6-carboxyfluorescein; 2′,4′,1,4,-tetrachlorofluorescein, and 2′,4′,5′,7′,1,4-hexachlorofluorescein. In certain aspects, the fluorescent label is selected from SYBR-Green, 6-carboxyfluorescein (“FAM”), TET, ROX, VIC™, and JOE. For example, in certain embodiments, labels are different fluorophores capable of emitting light at different, spectrally-resolvable wavelengths (e.g., 4-differently colored fluorophores); certain such labeled probes are known in the art and described above, and in U.S. Pat. No. 6,140,054. A dual labeled fluorescent probe that includes a reporter fluorophore and a quencher fluorophore is used in some embodiments. It will be appreciated that pairs of fluorophores are chosen that have distinct emission spectra so that they can be easily distinguished.

In further embodiments, labels are hybridization-stabilizing moieties which serve to enhance, stabilize, or influence hybridization of duplexes, e.g., intercalators and intercalating dyes (including, but not limited to, ethidium bromide and SYBR-Green), minor-groove binders, and cross-linking functional groups (see, e.g., Blackburn et al., eds. “DNA and RNA Structure” in Nucleic Acids in Chemistry and Biology (1996)).

In further aspects, methods relying on hybridization and/or ligation to quantify biomarker nucleic acid may be used including, but not limited to, oligonucleotide ligation (OLA) methods and methods that allow a distinguishable probe that hybridizes to the target nucleic acid sequence to be separated from an unbound probe. For example, HARP-like probes, as disclosed in U.S. Patent Application Publication No. 2006/0078894 may be used to measure the quantity of target nucleic acid. In such methods, after hybridization between a probe and the targeted nucleic acid, the probe is modified to distinguish the hybridized probe from the unhybridized probe. Thereafter, the probe may be amplified and/or detected. In general, a probe inactivation region comprises a subset of nucleotides within the target hybridization region of the probe. To reduce or prevent amplification or detection of a HARP probe that is not hybridized to its target nucleic acid, and thus allow detection of the target nucleic acid, a post-hybridization probe inactivation step is carried out using an agent which is able to distinguish between a HARP probe that is hybridized to its targeted nucleic acid sequence and the corresponding unhybridized HARP probe. The agent is able to inactivate or modify the unhybridized HARP probe such that it cannot be amplified.

In an additional embodiment of the method, a probe ligation reaction may be used to quantify target biomarker nucleic acid. In a Multiplex Ligation-dependent Probe Amplification (MLPA) technique, pairs of probes which hybridize immediately adjacent to each other on the target nucleic acid are ligated to each other only in the presence of the target nucleic acid. See Schouten et al., 30 NUCL. ACIDS RES. e57 (2002). In some aspects, MLPA probes have flanking PCR primer binding sites. MLPA probes can only be amplified if they have been ligated, thus allowing for detection and quantification of biomarkers.

Furthermore, a sample may also be analyzed by means of a microarray. Microarrays generally comprise solid substrates and have a generally planar surface, to which a capture reagent (also called an adsorbent or affinity reagent) is attached. Frequently, the surface of a microarray comprises a plurality of addressable locations, each of which has the capture reagent (e.g., miRNA probes specific for particular biomarkers) bound there. Many microarrays are described in the art. These include, for example, biochips produced by Asuragen, Inc. (Austin, Tex.); Affymetrix, Inc. (Santa Clara, Calif.); GenoSensor Corp. (Tempe, Ariz.); Invitrogen, Corp. (Carlsbad, Calif.); and Illumina, Inc. (San Diego, Calif.).

In particular embodiments, a method comprises the steps of (a) assaying gene expression levels of one or more AIM genes described herein (e.g., including a panel described herein) in a biological sample obtained from a patient; (b) calculating an immune signature value based on the assayed expression levels. In a specific embodiment, the assay step can comprise PCR amplification. In other embodiments, the method can further comprise generating a report summarizing the gene expression data and/or the immune signature values. In other embodiments, the method may further comprise recommending a particular treatment. For example, an immune signature that is determined to be low in comparison to other biomarkers/control levels indicates that the subject should be treated with epigenetic therapy followed immunotherapy, chemotherapy or some combination of therapy for the particular cancer. Alternatively, an immune signature that is determined to be high in comparison to other biomarkers/control levels indicates that the subject can be treated with immunotherapy (and optionally chemotherapy or some combination of therapy for the particular cancer). The methods listed above include all embodiments of the AIM panels described herein.

C. Detection by Electrochemicaluminescent Assay

In several embodiments, the biomarker biomarkers of the present invention may be detected by means of an electrochemicaluminescent assay developed by Meso Scale Discovery (Gaithersrburg, Md.). Electrochemiluminescence detection uses labels that emit light when electrochemically stimulated. Background signals are minimal because the stimulation mechanism (electricity) is decoupled from the signal (light). Labels are stable, non-radioactive and offer a choice of convenient coupling chemistries. They emit light at ˜620 nm, eliminating problems with color quenching. See U.S. Pat. No. 7,497,997; U.S. Pat. No. 7,491,540; U.S. Pat. No. 7,288,410; U.S. Pat. No. 7,036,946; U.S. Pat. No. 7,052,861; U.S. Pat. No. 6,977,722; U.S. Pat. No. 6,919,173; U.S. Pat. No. 6,673,533; U.S. Pat. No. 6,413,783; U.S. Pat. No. 6,362,011; U.S. Pat. No. 6,319,670; U.S. Pat. No. 6,207,369; U.S. Pat. No. 6,140,045; U.S. Pat. No. 6,090,545; and U.S. Pat. No. 5,866,434. See also U.S. Patent Applications Publication No. 2009/0170121; No. 2009/006339; No. 2009/0065357; No. 2006/0172340; No. 2006/0019319; No. 2005/0142033; No. 2005/0052646; No. 2004/0022677; No. 2003/0124572; No. 2003/0113713; No. 2003/0003460; No. 2002/0137234; No. 2002/0086335; and No. 2001/0021534.

D. Detection by Mass Spectrometry

In one aspect, the biomarkers of the present invention may be detected by mass spectrometry, a method that employs a mass spectrometer to detect gas phase ions. Examples of mass spectrometers are time-of-flight, magnetic sector, quadrupole filter, ion trap, ion cyclotron resonance, Orbitrap, hybrids or combinations of the foregoing, and the like.

In particular embodiments, the biomarkers of the present invention are detected using selected reaction monitoring (SRM) mass spectrometry techniques. Selected reaction monitoring (SRM) is a non-scanning mass spectrometry technique, performed on triple quadrupole-like instruments and in which collision-induced dissociation is used as a means to increase selectivity. In SRM experiments two mass analyzers are used as static mass filters, to monitor a particular fragment ion of a selected precursor ion. The specific pair of mass-over-charge (m/z) values associated to the precursor and fragment ions selected is referred to as a “transition” and can be written as parent m/z→fragment m/z (e.g. 673.5→534.3). Unlike common MS based proteomics, no mass spectra are recorded in a SRM analysis. Instead, the detector acts as counting device for the ions matching the selected transition thereby returning an intensity distribution over time. Multiple SRM transitions can be measured within the same experiment on the chromatographic time scale by rapidly toggling between the different precursor/fragment pairs (sometimes called multiple reaction monitoring, MRM). Typically, the triple quadrupole instrument cycles through a series of transitions and records the signal of each transition as a function of the elution time. The method allows for additional selectivity by monitoring the chromatographic coelution of multiple transitions for a given analyte. The terms SRM/MRM are occasionally used also to describe experiments conducted in mass spectrometers other than triple quadrupoles (e.g. in trapping instruments) where upon fragmentation of a specific precursor ion a narrow mass range is scanned in MS2 mode, centered on a fragment ion specific to the precursor of interest or in general in experiments where fragmentation in the collision cell is used as a means to increase selectivity. In this application the terms SRM and MRM or also SRM/MRM can be used interchangeably, since they both refer to the same mass spectrometer operating principle. As a matter of clarity, the term MRM is used throughout the text, but the term includes both SRM and MRM, as well as any analogous technique, such as e.g. highly-selective reaction monitoring, hSRM, LC-SRM or any other SRM/MRM-like or SRM/MRM-mimicking approaches performed on any type of mass spectrometer and/or, in which the peptides are fragmented using any other fragmentation method such as e.g. CAD (collision-activated dissociation (also known as CID or collision-induced dissociation), HCD (higher energy CID), ECD (electron capture dissociation), PD (photodissociation) or ETD (electron transfer dissociation).

In another specific embodiment, the mass spectrometric method comprises matrix assisted laser desorption/ionization time-of-flight (MALDI-TOF MS or MALDI-TOF). In another embodiment, method comprises MALDI-TOF tandem mass spectrometry (MALDI-TOF MS/MS). In yet another embodiment, mass spectrometry can be combined with another appropriate method(s) as may be contemplated by one of ordinary skill in the art. For example, MALDI-TOF can be utilized with trypsin digestion and tandem mass spectrometry as described herein.

In an alternative embodiment, the mass spectrometric technique comprises surface enhanced laser desorption and ionization or “SELDI,” as described, for example, in U.S. Pat. No. 6,225,047 and U.S. Pat. No. 5,719,060. Briefly, SELDI refers to a method of desorption/ionization gas phase ion spectrometry (e.g. mass spectrometry) in which an analyte (here, one or more of the biomarkers) is captured on the surface of a SELDI mass spectrometry probe. There are several versions of SELDI that may be utilized including, but not limited to, Affinity Capture Mass Spectrometry (also called Surface-Enhanced Affinity Capture (SEAC)), and Surface-Enhanced Neat Desorption (SEND) which involves the use of probes comprising energy absorbing molecules that are chemically bound to the probe surface (SEND probe). Another SELDI method is called Surface-Enhanced Photolabile Attachment and Release (SEPAR), which involves the use of probes having moieties attached to the surface that can covalently bind an analyte, and then release the analyte through breaking a photolabile bond in the moiety after exposure to light, e.g., to laser light (see, U.S. Pat. No. 5,719,060). SEPAR and other forms of SELDI are readily adapted to detecting a biomarker or biomarker panel, pursuant to the present invention.

In another mass spectrometry method, the biomarkers can be first captured on a chromatographic resin having chromatographic properties that bind the biomarkers. For example, one could capture the biomarkers on a cation exchange resin, such as CM Ceramic HyperD F resin, wash the resin, elute the biomarkers and detect by MALDI. Alternatively, this method could be preceded by fractionating the sample on an anion exchange resin before application to the cation exchange resin. In another alternative, one could fractionate on an anion exchange resin and detect by MALDI directly. In yet another method, one could capture the biomarkers on an immuno-chromatographic resin that comprises antibodies that bind the biomarkers, wash the resin to remove unbound material, elute the biomarkers from the resin and detect the eluted biomarkers by MALDI or by SELDI.

E. Other Methods for Detecting Biomarkers

The biomarkers of the present invention can be detected by other suitable methods. Detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

In specific embodiments, the biomarkers of the present invention are detected using nanotechnology including, for example, a nanowire. See, e.g., U.S. Pat. No. 8,323,466 and U.S. Patent Application Publication No. 20120258445 (NanoIVD, Inc. (Los Angeles, Calif.)).

Furthermore, a sample may also be analyzed by means of a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there. Protein biochips are biochips adapted for the capture of polypeptides. Many protein biochips are described in the art. These include, for example, protein biochips produced by Ciphergen Biosystems, Inc. (Fremont, Calif.), Invitrogen Corp. (Carlsbad, Calif.), Affymetrix, Inc. (Fremong, Calif.), Zyomyx (Hayward, Calif.), R&D Systems, Inc. (Minneapolis, Minn.), Biacore (Uppsala, Sweden) and Procognia (Berkshire, UK). Examples of such protein biochips are described in the following patents or published patent applications: U.S. Pat. No. 6,537,749; U.S. Pat. No. 6,329,209; U.S. Pat. No. 6,225,047; U.S. Pat. No. 5,242,828; PCT International Publication No. WO 00/56934; and PCT International Publication No. WO 03/048768.

### IV. DETERMINATION OF A PATIENT'S PROSTATE CANCER STATUS

The present invention relates to the use of biomarkers to diagnose prostate cancer. More specifically, the biomarkers of the present invention can be used in diagnostic tests to determine, qualify, and/or assess prostate cancer or status, for example, to diagnose prostate cancer (high or low grade, for example), in an individual, subject or patient. In particular embodiments, prostate cancer status can include determining a patient's prostate cancer status or prostate cancer status, for example, to diagnose prostate cancer, in an individual, subject or patient. More specifically, the biomarkers to be detected in diagnosing prostate cancer (e.g., high grade prostate cancer) include, but are not limited to, FGF2, PTGDS, GPX3, PTN, SERPINF1, ANGPTL2, SGCB, LTBP4, DST, MMP2, SRGN, DMD, FBLN1, ERAP1, FXYD6, LGALS3BP, ANG, GSN, SST, and DKK3. Other biomarkers known in the relevant art may be used in combination with the biomarkers described herein.

The levels of one or more of the biomarkers of Table 1 may be determined in the diagnostic and treatment methods. For example, the level(s) of one biomarker, two or more biomarkers, three or more biomarkers, four or more biomarkers, five or more biomarkers, six or more biomarkers, seven or more biomarkers, eight or more biomarkers, nine or more biomarkers, ten or more biomarkers, eleven or more biomarkers, twelve or more biomarkers, thirteen or more biomarkers, fourteen or more biomarkers, fifteen or more biomarkers, sixteen or more biomarkers, seventeen or more biomarkers, eighteen or more biomarkers, nineteen or more biomarkers, twenty or more biomarkers, etc., including the biomarkers in Table 1 and combinations thereof or any fraction thereof, may be determined and used in such methods. Determining levels of combinations of the biomarkers may allow greater sensitivity and specificity in diagnosing prostate cancer and aiding in the diagnosis of prostate cancer, and may allow better differentiation of prostate cancer from other prostate disorders (e.g. benign prostatic hypertrophy (BPH), prostatitis, etc.) or other cancers that may have similar or overlapping biomarkers to prostate cancer (as compared to a subject not having prostate cancer). For example, ratios of the levels of certain biomarkers (and non-biomarker compounds) in biological samples may allow greater sensitivity and specificity in diagnosing prostate cancer and aiding in the diagnosis of prostate cancer and may allow better differentiation of prostate cancer from other cancers or other disorders of the prostate that may have similar or overlapping biomarkers to prostate cancer (as compared to a subject not having prostate cancer).

After the level(s) of the one or more biomarkers in the sample are determined, the level(s) are compared to high grade prostate cancer-positive, low grade prostate cancer-positive and/or prostate cancer-negative reference control levels to aid in diagnosing or to diagnose whether the subject has high grade prostate cancer, low grade prostate cancer, or no prostate cancer. Levels of the one or more biomarkers in a sample matching the high grade prostate cancer-positive reference levels (e.g., levels that are the same as the reference levels, substantially the same as the reference levels, above and/or below the minimum and/or maximum of the reference levels, and/or within the range of the reference levels) are indicative of a diagnosis of high grade prostate cancer in the subject. Levels of the one or more biomarkers in a sample matching the low grade prostate cancer-positive reference levels (e.g., levels that are the same as the reference levels, substantially the same as the reference levels, above and/or below the minimum and/or maximum of the reference levels, and/or within the range of the reference levels) are indicative of a diagnosis of low grade prostate cancer in the subject. Levels of the one or more biomarkers in a sample matching the prostate cancer-negative reference levels (e.g., levels that are the same as the reference levels, substantially the same as the reference levels, above and/or below the minimum and/or maximum of the reference levels, and/or within the range of the reference levels) are indicative of a diagnosis of no prostate cancer in the subject. In addition, levels of the one or more biomarkers that are differentially present (especially at a level that is statistically significant) in the sample as compared to prostate cancer-negative reference levels are indicative of a diagnosis of prostate cancer in the subject. Levels of the one or more biomarkers that are differentially present (especially at a level that is statistically significant) in the sample as compared to prostate cancer-positive reference levels are indicative of a diagnosis of no prostate cancer in the subject.

The level(s) of the one or more biomarkers may be compared to prostate cancer-positive (high and/or low) and/or prostate cancer-negative reference levels using various techniques, including a simple comparison (e.g., a manual comparison) of the level(s) of the one or more biomarkers in the biological sample to prostate cancer-positive and/or prostate cancer-negative reference levels. The level(s) of the one or more biomarkers in the biological sample may also be compared to prostate cancer-positive and/or prostate cancer-negative reference levels using one or more statistical analyses, including those methods described herein (e.g., t-test, Welch's T-test, Wilcoxon's rank sum test, random forest).

In addition, the biological samples may be analyzed to determine the level(s) of one or more non-biomarker compounds. The level(s) of such non-biomarker compounds may also allow differentiation of prostate cancer from other prostate disorders that may have similar or overlapping biomarkers to prostate cancer (as compared to a subject not having a prostate disorder). For example, a known non-biomarker compound present in biological samples of subjects having prostate cancer and subjects not having prostate cancer could be monitored to verify a diagnosis of prostate cancer as compared to a diagnosis of another prostate disorder when biological samples from subjects having the prostate disorder do not have the non-biomarker compound.

Similarly, the methods of diagnosing (or aiding in diagnosing) whether a subject has prostate cancer, as well as methods for treating, may also be conducted specifically to diagnose (or aid in diagnosing) whether a subject has less aggressive prostate cancer and/or high aggressive prostate cancer (and/or to decide how to treat such subjects). Such methods comprise obtaining a biological sample from a subject, performing an assay on the biological sample to measure or determine the level(s) of one or more biomarkers of high aggressive prostate cancer in the sample and identifying the subject as having high aggressive prostate cancer (or not) based on a comparison of the level(s) of the one or more biomarkers in the sample to high aggressive prostate cancer-positive and/or high aggressive prostate cancer-negative reference levels (and/or less aggressive prostate cancer-positive and/or less aggressive prostate cancer-negative reference levels) in order to treat or diagnose (or aid in the diagnosis of) whether the subject has less aggressive prostate cancer (or high aggressive prostate cancer). Biomarker specific for high grade prostate cancer are listed in Table 1.

The identification of biomarkers for distinguishing more or high aggressive prostate cancer versus less aggressive prostate cancer allows less aggressive prostate cancer and aggressive prostate cancer to be distinguished in patients. The subjects can then be treated appropriately, with those subjects having more aggressive prostate cancer undergoing more aggressive treatment than those subjects with less aggressive prostate cancer including radical prostatectomy, radiation therapy, hormone therapy and/or chemotherapy. A method of distinguishing less aggressive prostate cancer from more aggressive prostate cancer in a subject having prostate cancer comprises obtaining a biological sample from a subject; performing an assay on the biological sample to determine the level(s) in the sample of one or more biomarkers of high aggressive prostate cancer that distinguish over less aggressive prostate cancer, and (2) identifying the subject as having high aggressive prostate cancer based on a comparison of the level(s) of the one or more biomarkers in the sample to high aggressive prostate cancer-positive reference levels that distinguish over less aggressive prostate cancer of the one or more biomarkers and/or less aggressive prostate cancer-positive reference levels that distinguish over high aggressive prostate cancer in order to determine whether the subject has less aggressive or high aggressive prostate cancer and how to treat such subject. The one or more biomarkers that are used are selected from Table 1 and combinations thereof.

Any suitable method may be used to perform the assay on the biological sample in order to determine the level(s) of the one or more biomarkers in the sample. Suitable methods include chromatography (e.g., HPLC, gas chromatography, liquid chromatography), mass spectrometry (e.g., MS, MS-MS), enzyme-linked immunosorbent assay (ELISA), antibody linkage, other immunochemical techniques, and combinations thereof. Further, the level(s) of the one or more biomarkers may be measured indirectly, for example, by using an assay that measures the level of a compound (or compounds) that correlates with the level of the biomarker(s) that are desired to be measured.

After the level(s) of the one or more biomarkers in the sample are determined/measured, the level(s) are compared to one or more reference controls including, for example, high aggressive prostate cancer-positive reference levels that distinguish over less aggressive prostate cancer (and/or less aggressive prostate cancer-positive reference levels that distinguish over high aggressive prostate cancer-negative) to determine whether the subject has less aggressive or high aggressive prostate cancer. Levels of the one or more biomarkers in a sample matching the less aggressive prostate cancer-positive reference levels that distinguish over high aggressive prostate cancer (e.g., levels that are the same as the reference levels, substantially the same as the reference levels, above and/or below the minimum and/or maximum of the reference levels, and/or within the range of the reference levels) are indicative of less aggressive prostate cancer in the subject. Levels of the one or more biomarkers in a sample matching the high aggressive prostate cancer-positive reference levels that distinguish over low aggressive prostate cancer (e.g., levels that are the same as the reference levels, substantially the same as the reference levels, above and/or below the minimum and/or maximum of the reference levels, and/or within the range of the reference levels) are indicative of high-aggressive prostate cancer in the subject. If the level(s) of the one or more biomarkers are more similar to the less aggressive prostate cancer-positive reference levels that distinguish over high aggressive prostate cancer (or less similar to the high aggressive prostate cancer-positive reference levels), then the results are indicative of less aggressive prostate cancer in the subject. If the level(s) of the one or more biomarkers are more similar to the high aggressive prostate cancer-positive reference levels that distinguish over less aggressive prostate cancer (or less similar to the less aggressive prostate cancer-positive reference levels), then the results are indicative of high aggressive prostate cancer in the subject.

The level(s) of the one or more biomarkers may be compared to high aggressive prostate cancer-positive reference levels that distinguish over less aggressive prostate cancer (and/or less aggressive prostate cancer-positive reference levels that distinguish over high aggressive prostate cancer) using various techniques, including a simple comparison (e.g., a manual comparison) of the level(s) of the one or more biomarkers in the biological sample to less aggressive prostate cancer-positive and/or high aggressive prostate cancer-positive reference levels. The level(s) of the one or more biomarkers in the biological sample may also be compared to less aggressive prostate cancer-positive reference levels that distinguish over high aggressive prostate cancer and/or high aggressive prostate cancer-positive reference levels that distinguish over less aggressive prostate cancer using one or more statistical analyses, including those methods described herein (e.g., t-test, Welch's T-test, Wilcoxon's rank sum test, random forest).

In addition, the biological samples may be analyzed to determine the level(s) of one or more non-biomarker compounds. The level(s) of such non-biomarker compounds may also allow differentiation of less aggressive prostate cancer from high aggressive prostate cancer.

The levels of one or more of the biomarkers of Table 1 may be determined in the diagnostic and treatment methods. For example, the level(s) of one biomarker, two or more biomarkers, three or more biomarkers, four or more biomarkers, five or more biomarkers, six or more biomarkers, seven or more biomarkers, eight or more biomarkers, nine or more biomarkers, ten or more biomarkers, eleven or more biomarkers, twelve or more biomarkers, thirteen or more biomarkers, fourteen or more biomarkers, fifteen or more biomarkers, sixteen or more biomarkers, seventeen or more biomarkers, eighteen or more biomarkers, nineteen or more biomarkers, twenty or more biomarkers, etc., including the biomarkers in Table 1 and combinations thereof or any fraction thereof, may be determined and used in such methods. Determining levels of combinations of the biomarkers may allow greater sensitivity and specificity in diagnosing prostate cancer and aiding in the diagnosis of prostate cancer, and may allow better differentiation of prostate cancer from other prostate disorders (e.g. benign prostatic hypertrophy (BPH), prostatitis, etc.) or other cancers that may have similar or overlapping biomarkers to prostate cancer (as compared to a subject not having prostate cancer). For example, ratios of the levels of certain biomarkers (and non-biomarker compounds) in biological samples may allow greater sensitivity and specificity in diagnosing prostate cancer and aiding in the diagnosis of prostate cancer and may allow better differentiation of prostate cancer from other cancers or other disorders of the prostate that may have similar or overlapping biomarkers to prostate cancer (as compared to a subject not having prostate cancer).

After the level(s) of the one or more biomarkers in the sample are determined, the level(s) are compared to high grade prostate cancer-positive, low grade prostate cancer-positive and/or prostate cancer-negative reference control levels to aid in diagnosing or to diagnose whether the subject has high grade prostate cancer, low grade prostate cancer, or no prostate cancer. Levels of the one or more biomarkers in a sample matching the high grade prostate cancer-positive reference levels (e.g., levels that are the same as the reference levels, substantially the same as the reference levels, above and/or below the minimum and/or maximum of the reference levels, and/or within the range of the reference levels) are indicative of a diagnosis of high grade prostate cancer in the subject. Levels of the one or more biomarkers in a sample matching the low grade prostate cancer-positive reference levels (e.g., levels that are the same as the reference levels, substantially the same as the reference levels, above and/or below the minimum and/or maximum of the reference levels, and/or within the range of the reference levels) are indicative of a diagnosis of low grade prostate cancer in the subject. Levels of the one or more biomarkers in a sample matching the prostate cancer-negative reference levels (e.g., levels that are the same as the reference levels, substantially the same as the reference levels, above and/or below the minimum and/or maximum of the reference levels, and/or within the range of the reference levels) are indicative of a diagnosis of no prostate cancer in the subject. In addition, levels of the one or more biomarkers that are differentially present (especially at a level that is statistically significant) in the sample as compared to prostate cancer-negative reference levels are indicative of a diagnosis of prostate cancer in the subject. Levels of the one or more biomarkers that are differentially present (especially at a level that is statistically significant) in the sample as compared to prostate cancer-positive reference levels are indicative of a diagnosis of no prostate cancer in the subject.

The level(s) of the one or more biomarkers may be compared to prostate cancer-positive (high and/or low) and/or prostate cancer-negative reference levels using various techniques, including a simple comparison (e.g., a manual comparison) of the level(s) of the one or more biomarkers in the biological sample to prostate cancer-positive and/or prostate cancer-negative reference levels. The level(s) of the one or more biomarkers in the biological sample may also be compared to prostate cancer-positive and/or prostate cancer-negative reference levels using one or more statistical analyses, including those methods described herein (e.g., t-test, Welch's T-test, Wilcoxon's rank sum test, random forest).

In addition, the biological samples may be analyzed to determine the level(s) of one or more non-biomarker compounds. The level(s) of such non-biomarker compounds may also allow differentiation of prostate cancer from other prostate disorders that may have similar or overlapping biomarkers to prostate cancer (as compared to a subject not having a prostate disorder). For example, a known non-biomarker compound present in biological samples of subjects having prostate cancer and subjects not having prostate cancer could be monitored to verify a diagnosis of prostate cancer as compared to a diagnosis of another prostate disorder when biological samples from subjects having the prostate disorder do not have the non-biomarker compound.

Similarly, the methods of diagnosing (or aiding in diagnosing) whether a subject has prostate cancer, as well as methods for treating, may also be conducted specifically to diagnose (or aid in diagnosing) whether a subject has less aggressive prostate cancer and/or high aggressive prostate cancer (and/or to decide how to treat such subjects). Such methods comprise obtaining a biological sample from a subject, performing an assay on the biological sample to measure or determine the level(s) of one or more biomarkers of high aggressive prostate cancer in the sample and identifying the subject as having high aggressive prostate cancer (or not) based on a comparison of the level(s) of the one or more biomarkers in the sample to high aggressive prostate cancer-positive and/or high aggressive prostate cancer-negative reference levels (and/or less aggressive prostate cancer-positive and/or less aggressive prostate cancer-negative reference levels) in order to treat or diagnose (or aid in the diagnosis of) whether the subject has less aggressive prostate cancer (or high aggressive prostate cancer). Biomarker specific for high grade prostate cancer are listed in Table 1.

The identification of biomarkers for distinguishing more or high aggressive prostate cancer versus less aggressive prostate cancer allows less aggressive prostate cancer and aggressive prostate cancer to be distinguished in patients. The subjects can then be treated appropriately, with those subjects having more aggressive prostate cancer undergoing more aggressive treatment than those subjects with less aggressive prostate cancer including radical prostatectomy, radiation therapy, hormone therapy and/or chemotherapy. A method of distinguishing less aggressive prostate cancer from more aggressive prostate cancer in a subject having prostate cancer comprises obtaining a biological sample from a subject; performing an assay on the biological sample to determine the level(s) in the sample of one or more biomarkers of high aggressive prostate cancer that distinguish over less aggressive prostate cancer, and (2) identifying the subject as having high aggressive prostate cancer based on a comparison of the level(s) of the one or more biomarkers in the sample to high aggressive prostate cancer-positive reference levels that distinguish over less aggressive prostate cancer of the one or more biomarkers and/or less aggressive prostate cancer-positive reference levels that distinguish over high aggressive prostate cancer in order to determine whether the subject has less aggressive or high aggressive prostate cancer and how to treat such subject. The one or more biomarkers that are used are selected from Table 1 and combinations thereof.

Any suitable method may be used to perform the assay on the biological sample in order to determine the level(s) of the one or more biomarkers in the sample. Suitable methods include chromatography (e.g., HPLC, gas chromatography, liquid chromatography), mass spectrometry (e.g., MS, MS-MS), enzyme-linked immunosorbent assay (ELISA), antibody linkage, other immunochemical techniques, and combinations thereof. Further, the level(s) of the one or more biomarkers may be measured indirectly, for example, by using an assay that measures the level of a compound (or compounds) that correlates with the level of the biomarker(s) that are desired to be measured.

After the level(s) of the one or more biomarkers in the sample are determined/measured, the level(s) are compared to one or more reference controls including, for example, high aggressive prostate cancer-positive reference levels that distinguish over less aggressive prostate cancer (and/or less aggressive prostate cancer-positive reference levels that distinguish over high aggressive prostate cancer-negative) to determine whether the subject has less aggressive or high aggressive prostate cancer. Levels of the one or more biomarkers in a sample matching the less aggressive prostate cancer-positive reference levels that distinguish over high aggressive prostate cancer (e.g., levels that are the same as the reference levels, substantially the same as the reference levels, above and/or below the minimum and/or maximum of the reference levels, and/or within the range of the reference levels) are indicative of less aggressive prostate cancer in the subject. Levels of the one or more biomarkers in a sample matching the high aggressive prostate cancer-positive reference levels that distinguish over low aggressive prostate cancer (e.g., levels that are the same as the reference levels, substantially the same as the reference levels, above and/or below the minimum and/or maximum of the reference levels, and/or within the range of the reference levels) are indicative of high-aggressive prostate cancer in the subject. If the level(s) of the one or more biomarkers are more similar to the less aggressive prostate cancer-positive reference levels that distinguish over high aggressive prostate cancer (or less similar to the high aggressive prostate cancer-positive reference levels), then the results are indicative of less aggressive prostate cancer in the subject. If the level(s) of the one or more biomarkers are more similar to the high aggressive prostate cancer-positive reference levels that distinguish over less aggressive prostate cancer (or less similar to the less aggressive prostate cancer-positive reference levels), then the results are indicative of high aggressive prostate cancer in the subject.

The level(s) of the one or more biomarkers may be compared to high aggressive prostate cancer-positive reference levels that distinguish over less aggressive prostate cancer (and/or less aggressive prostate cancer-positive reference levels that distinguish over high aggressive prostate cancer) using various techniques, including a simple comparison (e.g., a manual comparison) of the level(s) of the one or more biomarkers in the biological sample to less aggressive prostate cancer-positive and/or high aggressive prostate cancer-positive reference levels. The level(s) of the one or more biomarkers in the biological sample may also be compared to less aggressive prostate cancer-positive reference levels that distinguish over high aggressive prostate cancer and/or high aggressive prostate cancer-positive reference levels that distinguish over less aggressive prostate cancer using one or more statistical analyses, including those methods described herein (e.g., t-test, Welch's T-test, Wilcoxon's rank sum test, random forest).

In addition, the biological samples may be analyzed to determine the level(s) of one or more non-biomarker compounds. The level(s) of such non-biomarker compounds may also allow differentiation of less aggressive prostate cancer from high aggressive prostate cancer.

A. Biomarker Panels

The biomarkers of the present invention can be used in diagnostic tests to assess, determine, and/or qualify (used interchangeably herein) prostate cancer status in a patient. The phrase “prostate cancer status” includes any distinguishable manifestation of the condition, including not having prostate cancer. For example, prostate cancer status includes, without limitation, the presence or absence of prostate cancer in a patient, the risk of developing prostate cancer, the stage or severity of prostate cancer, the progress of prostate cancer (e.g., progress of prostate cancer over time) and the effectiveness or response to treatment of prostate cancer (e.g., clinical follow up and surveillance of prostate cancer after treatment). Based on this status, further procedures may be indicated, including additional diagnostic tests or therapeutic procedures or regimens.

These and other biomarkers are disclosed herein, and it is understood that when combinations, subsets, interactions, groups, etc., of these biomarkers are disclosed that while specific reference of each various individual and collective combinations and permutation of these compounds may not be explicitly disclosed, each is specifically contemplated and described herein. Thus, if a panel of biomarkers A, B, and C are disclosed as well as a class of biomarkers D, E, and F and an example of a combination panel A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of using the disclosed biomarkers. Thus, if there are a variety of additional steps that can be performed, it is understood that each of these additional steps can be performed with any specific embodiment or combination of embodiments of the disclosed methods.

Accordingly, in one embodiment, the panel comprises one or more of FGF2, PTGDS, GPX3, PTN, SERPINF1, ANGPTL2, SGCB, LTBP4, DST, MMP2, SRGN, DMD, FBLN1, ERAP1, FXYD6, LGALS3BP, ANG, GSN, SST, and DKK3. The foregoing includes, for example, combinations of 2, 3, 4, 5, 6, 7, 8, 9, 10, 11, 12, 13, 14, 15, 16, 17, 18, and 20 biomarkers.

The power of a diagnostic test to correctly predict status is commonly measured as the sensitivity of the assay, the specificity of the assay or the area under a receiver operated characteristic (“ROC”) curve. Sensitivity is the percentage of true positives that are predicted by a test to be positive, while specificity is the percentage of true negatives that are predicted by a test to be negative. An ROC curve provides the sensitivity of a test as a function of 1-specificity. The greater the area under the ROC curve, the more powerful the predictive value of the test. Other useful measures of the utility of a test are positive predictive value and negative predictive value. Positive predictive value is the percentage of people who test positive that are actually positive. Negative predictive value is the percentage of people who test negative that are actually negative.

In particular embodiments, the biomarker panels of the present invention may show a statistical difference in different prostate cancer statuses of at least p<0.05, p<10⁻², p<10⁻³, p<10⁻⁴ or p<10⁻⁵. Diagnostic tests that use these biomarkers may show an ROC of at least 0.6, at least about 0.7, at least about 0.8, or at least about 0.9.

The biomarkers can be differentially present in UI (NC or non-prostate cancer) and prostate cancer, or, for example, low grade vs. high grade prostate cancer, and, therefore, are useful in aiding in the determination of prostate cancer status. In certain embodiments, the biomarkers are measured in a patient sample using the methods described herein and compared, for example, to predefined biomarker levels/ratios and correlated to prostate cancer status. In particular embodiments, the measurement(s) may then be compared with a relevant diagnostic amount(s), cut-off(s), or multivariate model scores that distinguish a positive prostate cancer status from a negative prostate cancer status. The diagnostic amount(s) represents a measured amount of a biomarker(s) above which or below which a patient is classified as having a particular prostate cancer status. For example, if the biomarker(s) is/are up-regulated compared to normal during prostate cancer, then a measured amount(s) above the diagnostic cutoff(s) provides a diagnosis of prostate cancer. Alternatively, if the biomarker(s) is/are down-regulated during prostate cancer, then a measured amount(s) at or below the diagnostic cutoff(s) provides a diagnosis of non-prostate cancer. As is well understood in the art, by adjusting the particular diagnostic cut-off(s) used in an assay, one can increase sensitivity or specificity of the diagnostic assay depending on the preference of the diagnostician. In particular embodiments, the particular diagnostic cut-off can be determined, for example, by measuring the amount of biomarkers in a statistically significant number of samples from patients with the different prostate cancer statuses, and drawing the cut-off to suit the desired levels of specificity and sensitivity.

In other embodiments, ratios of post-translationally modified biomarkers to the corresponding unmodified biomarkers are useful in aiding in the determination of prostate cancer status. In certain embodiments, the biomarker ratios are indicative of diagnosis. In other embodiments, a biomarker ratio can be compared to another biomarker ratio in the same sample or to a set of biomarker ratios from a control or reference sample.

Indeed, as the skilled artisan will appreciate there are many ways to use the measurements of two or more biomarkers in order to improve the diagnostic question under investigation. In a quite simple, but nonetheless often effective approach, a positive result is assumed if a sample is positive for at least one of the markers investigated.

Furthermore, in certain embodiments, the values measured for markers of a biomarker panel are mathematically combined and the combined value is correlated to the underlying diagnostic question. Biomarker values may be combined by any appropriate state of the art mathematical method. Well-known mathematical methods for correlating a marker combination to a disease status employ methods like discriminant analysis (DA) (e.g., linear-, quadratic-, regularized-DA), Discriminant Functional Analysis (DFA), Kernel Methods (e.g., SVM), Multidimensional Scaling (MDS), Nonparametric Methods (e.g., k-Nearest-Neighbor Classifiers), PLS (Partial Least Squares), Tree-Based Methods (e.g., Logic Regression, CART, Random Forest Methods, Boosting/Bagging Methods), Generalized Linear Models (e.g., Logistic Regression), Principal Components based Methods (e.g., SIMCA), Generalized Additive Models, Fuzzy Logic based Methods, Neural Networks and Genetic Algorithms based Methods. The skilled artisan will have no problem in selecting an appropriate method to evaluate a biomarker combination of the present invention. In one embodiment, the method used in a correlating a biomarker combination of the present invention, e.g. to diagnose prostate cancer, is selected from DA (e.g., Linear-, Quadratic-, Regularized Discriminant Analysis), DFA, Kernel Methods (e.g., SVM), MDS, Nonparametric Methods (e.g., k-Nearest-Neighbor Classifiers), PLS (Partial Least Squares), Tree-Based Methods (e.g., Logic Regression, CART, Random Forest Methods, Boosting Methods), or Generalized Linear Models (e.g., Logistic Regression), and Principal Components Analysis. Details relating to these statistical methods are found in the following references: Ruczinski et al., 12 J. OF COMPUTATIONAL AND GRAPHICAL STATISTICS 475-511 (2003); Friedman, J. H., 84 J. OF THE AMERICAN STATISTICAL ASSOCIATION 165-75 (1989); Hastie, Trevor, Tibshirani, Robert, Friedman, Jerome, The Elements of Statistical Learning, Springer Series in Statistics (2001); Breiman, L., Friedman, J. H., Olshen, R. A., Stone, C. J. Classification and regression trees, California: Wadsworth (1984); Breiman, L., 45 MACHINE LEARNING 5-32 (2001); Pepe, M. S., The Statistical Evaluation of Medical Tests for Classification and Prediction, Oxford Statistical Science Series, 28 (2003); and Duda, R. O., Hart, P. E., Stork, D. G., Pattern Classification, Wiley Interscience, 2nd Edition (2001).

B. Determining Risk of Developing Prostate Cancer

In a specific embodiment, the present invention provides methods for determining the risk of developing prostate cancer in a patient. Biomarker percentages, ratios, amounts or patterns are characteristic of various risk states, e.g., high, medium or low. The risk of developing prostate cancer is determined by measuring the relevant biomarker(s) and then either submitting them to a classification algorithm or comparing them with a reference amount, i.e., a predefined level or pattern of biomarker(s) that is associated with the particular risk level.

C. Determining Prostate Cancer Severity

In another embodiment, the present invention provides methods for determining the severity of prostate cancer in a patient. Each grade or stage of prostate cancer likely has a characteristic level of a biomarker or relative levels/ratios of a set of biomarkers (a pattern or ratio). The severity of prostate cancer is determined by measuring the relevant biomarker(s) and then either submitting them to a classification algorithm or comparing them with a reference amount, i.e., a predefined level or pattern of biomarker(s) that is associated with the particular stage.

D. Determining Prostate Cancer Prognosis

In one embodiment, the present invention provides methods for determining the course of prostate cancer in a patient. Prostate cancer course refers to changes in prostate cancer status over time, including prostate cancer progression (worsening) and prostate cancer regression (improvement). Over time, the amount or relative amount (e.g., the pattern or ratio) of the biomarkers changes. For example, biomarker “X” may be increased with prostate cancer, while biomarker “Y” may be decreased with prostate cancer. Therefore, the trend of these biomarkers, either increased or decreased over time toward prostate cancer or non-prostate cancer indicates the course of the condition. Accordingly, this method involves measuring the level of one or more biomarkers in a patient at least two different time points, e.g., a first time and a second time, and comparing the change, if any. The course of prostate cancer is determined based on these comparisons.

E. Patient Management

In certain embodiments of the methods of qualifying prostate cancer status, the methods further comprise managing patient treatment based on the status. Such management includes the actions of the physician or clinician subsequent to determining prostate cancer status. For example, if a physician makes a diagnosis of prostate cancer, then a certain regime of monitoring would follow. An assessment of the course of prostate cancer using the methods of the present invention may then require a certain prostate cancer therapy regimen. Alternatively, a diagnosis of non-prostate cancer might be followed with further testing to determine a specific disease that the patient might be suffering from. Also, further tests may be called for if the diagnostic test gives an inconclusive result on prostate cancer status.

F. Determining Therapeutic Efficacy of Pharmaceutical Drug

In another embodiment, the present invention provides methods for determining the therapeutic efficacy of a pharmaceutical drug. These methods are useful in performing clinical trials of the drug, as well as monitoring the progress of a patient on the drug. Therapy or clinical trials involve administering the drug in a particular regimen. The regimen may involve a single dose of the drug or multiple doses of the drug over time. The doctor or clinical researcher monitors the effect of the drug on the patient or subject over the course of administration. If the drug has a pharmacological impact on the condition, the amounts or relative amounts (e.g., the pattern, profile or ratio) of one or more of the biomarkers of the present invention may change toward a non-prostate cancer profile. Therefore, one can follow the course of one or more biomarkers in the patient during the course of treatment. Accordingly, this method involves measuring one or more biomarkers in a patient receiving drug therapy, and correlating the biomarker levels/ratios with the prostate cancer status of the patient (e.g., by comparison to predefined levels/ratios of the biomarkers that correspond to different prostate cancer statuses). One embodiment of this method involves determining the levels/ratios of one or more biomarkers for at least two different time points during a course of drug therapy, e.g., a first time and a second time, and comparing the change in levels/ratios of the biomarkers, if any. For example, the levels/ratios of one or more biomarkers can be measured before and after drug administration or at two different time points during drug administration. The effect of therapy is determined based on these comparisons. If a treatment is effective, then the level/ratio of one or more biomarkers will trend toward normal, while if treatment is ineffective, the level/ratio of one or more biomarkers will trend toward prostate cancer indications.

G. Generation of Classification Algorithms for Qualifying Prostate Cancer Status

In some embodiments, data that are generated using samples such as “known samples” can then be used to “train” a classification model. A “known sample” is a sample that has been pre-classified. The data that are used to form the classification model can be referred to as a “training data set.” The training data set that is used to form the classification model may comprise raw data or pre-processed data. Once trained, the classification model can recognize patterns in data generated using unknown samples. The classification model can then be used to classify the unknown samples into classes. This can be useful, for example, in predicting whether or not a particular biological sample is associated with a certain biological condition (e.g., diseased versus non-diseased).

Classification models can be formed using any suitable statistical classification or learning method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000, the teachings of which are incorporated by reference.

In supervised classification, training data containing examples of known categories are presented to a learning mechanism, which learns one or more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART), artificial neural networks such as back propagation networks, discriminant analyses (e.g., Bayesian classifier or Fischer analysis), logistic classifiers, and support vector classifiers (support vector machines).

Another supervised classification method is a recursive partitioning process. Recursive partitioning processes use recursive partitioning trees to classify data derived from unknown samples. Further details about recursive partitioning processes are provided in U.S. Patent Application No. 2002 0138208 A1 to Paulse et al., “Method for analyzing mass spectra.”

In other embodiments, the classification models that are created can be formed using unsupervised learning methods. Unsupervised classification attempts to learn classifications based on similarities in the training data set, without pre-classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

Learning algorithms asserted for use in classifying biological information are described, for example, in PCT International Publication No. WO 01/31580 (Barnhill et al., “Methods and devices for identifying patterns in biological systems and methods of use thereof”), U.S. Patent Application Publication No. 2002/0193950 (Gavin et al. “Method or analyzing mass spectra”), U.S. Patent Application Publication No. 2003/0004402 (Hitt et al., “Process for discriminating between biological states based on hidden patterns from biological data”), and U.S. Patent Application Publication No. 2003/0055615 (Zhang and Zhang, “Systems and methods for processing biological expression data”).

The classification models can be formed on and used on any suitable digital computer. Suitable digital computers include micro, mini, or large computers using any standard or specialized operating system, such as a UNIX, Windows® or Linux™ based operating system. In embodiments utilizing a mass spectrometer, the digital computer that is used may be physically separate from the mass spectrometer that is used to create the spectra of interest, or it may be coupled to the mass spectrometer.

The training data set and the classification models according to embodiments of the invention can be embodied by computer code that is executed or used by a digital computer. The computer code can be stored on any suitable computer readable media including optical or magnetic disks, sticks, tapes, etc., and can be written in any suitable computer programming language including R, C, C++, visual basic, etc.

The learning algorithms described above are useful both for developing classification algorithms for the biomarkers already discovered, and for finding new biomarker biomarkers. The classification algorithms, in turn, form the base for diagnostic tests by providing diagnostic values (e.g., cut-off points) for biomarkers used singly or in combination.

### V. KITS FOR THE DETECTION OF PROSTATE CANCER BIOMARKERS

In another aspect, the present invention provides kits for qualifying prostate cancer status, which kits are used to detect the biomarkers described herein. In particular embodiments, the kit is provided as an ELISA kit comprising antibodies to the biomarker(s) of the present invention. In a specific embodiment, the antibodies specifically bind to a protein biomarker, which biomarkers include one or more of FGF2, PTGDS, GPX3, PTN, SERPINF1, ANGPTL2, SGCB, LTBP4, DST, MMP2, SRGN, DMD, FBLN1, ERAP1, FXYD6, LGALS3BP, ANG, GSN, SST, and DKK3.

The ELISA kit may comprise a solid support, such as a chip, microtiter plate (e.g., a 96-well plate), bead, or resin having biomarker capture reagents attached thereon. The kit may further comprise a means for detecting the biomarker(s), such as antibodies, and a secondary antibody-signal complex such as horseradish peroxidase (HRP)-conjugated goat anti-rabbit IgG antibody and tetramethyl benzidine (TMB) as a substrate for HRP.

The kit for qualifying prostate cancer status may be provided as an immuno-chromatography strip comprising a membrane on which the antibodies are immobilized, and a means for detecting, e.g., gold particle bound antibodies, where the membrane, includes NC membrane and PVDF membrane. The kit may comprise a plastic plate on which a sample application pad, gold particle bound antibodies temporally immobilized on a glass fiber filter, a nitrocellulose membrane on which antibody bands and a secondary antibody band are immobilized and an absorbent pad are positioned in a serial manner, so as to keep continuous capillary flow of blood serum.

In certain embodiments, a patient can be diagnosed or identified by adding blood or blood serum from the patient to the kit and detecting the relevant biomarker(s) conjugated with antibodies, specifically, by a method which comprises the steps of: (i) collecting blood or blood serum from the patient; (ii) separating blood serum from the patient's blood; (iii) adding the blood serum from patient to a diagnostic kit; and, (iv) detecting the biomarker(s) conjugated with antibodies. In this method, the antibodies are brought into contact with the patient's blood. If the biomarkers are present in the sample, the antibodies will bind to the sample, or a portion thereof, to create an antibody:biomarker complex, which can then be detected/quantified and further compared to reference levels to identify the patient has having high grade (or low grade) prostate cancer. In other kit and diagnostic embodiments, blood or blood serum need not be collected from the patient (i.e., it is already collected). Moreover, in other embodiments, the sample may comprise a tissue sample, urine or a clinical sample.

The kit can also comprise a washing solution or instructions for making a washing solution, in which the combination of the capture reagents and the washing solution allows capture of the biomarkers on the solid support for subsequent detection by, e.g., antibodies or mass spectrometry. In a further embodiment, a kit can comprise instructions for suitable operational parameters in the form of a label or separate insert. For example, the instructions may inform a consumer about how to collect the sample, how to wash the probe or the particular biomarkers to be detected, etc. In yet another embodiment, the kit can comprise one or more containers with biomarker samples, to be used as standard(s) for calibration.

In another specific embodiment, the kit is provided as a PCR kit comprising primers that specifically bind to one or more of the nucleic acid biomarkers described herein. One of ordinary skill in the art can design primers the specifically bind and amplify the target biomarkers described herein including FGF2, PTGDS, GPX3, PTN, SERPINF1, ANGPTL2, SGCB, LTBP4, DST, MMP2, SRGN, DMD, FBLN1, ERAP1, FXYD6, LGALS3BP, ANG, GSN, SST, and DKK3. The kit can further comprise substrates and other reagents necessary for conducting PCR (e.g., quantitative real-time PCR). The kit can be configured to conduct singleplex or multiplex PCR. The kit can further comprise instructions for carrying out the PCR reaction(s). In specific embodiments, the biological sample obtained from a subject may be manipulated to extract nucleic acid. In a further embodiment, the nucleic acids are contacted with primers that specifically bind the target biomarkers to form a primer:biomarker complex. The complexes can then be amplified and detected/quantified/measured to determine the levels of one or more biomarkers. The subject can then be identified as having high grade/high aggressive (or low grade/less aggressive) prostate cancer based on a comparison of the measured levels of one or more biomarkers to one or more reference controls.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

Widespread use of PSA testing has resulted in the earlier detection of prostate cancer and has contributed to the 30% decrease in prostate cancer mortality since its peak in 1991. However, due to the lead time associated with PSA based screening, and its relative inability to distinguish patients with indolent and life threatening cancers, there is growing concern of over-diagnosis and over-treatment in screened populations. Indeed, the majority of patients currently diagnosed with prostate cancer have low grade (Gleason pattern 3), good risk disease and, if not upgraded at the time of surgery, would likely have low cancer specific mortality rates even without intervention. Thus, a critical goal in prostate cancer screening is to identify only those men who have clinically significant disease which requires treatment.

Presently used parameters to identify clinically significant prostate cancer include PSA at diagnosis, tumor stage and tumor grade on biopsy and/or at radical prostatectomy. Among these features, the most potent forecaster of metastatic ability and prostate cancer specific mortality is tumor grade. The modern grading system was described by the pathologist Gleason over three decades ago and most recently was updated in 2005. In this system, prostate cancer architecture is evaluated at low power and a score is applied to the most common and second most common architectural types (between 1 and 5, progressing from good to adverse). Retrospective studies and randomized controlled trials have demonstrated that prostate cancer containing only Gleason pattern 3 disease (Gleason sum≦6) have uniformly favorable outcomes, even when managed expectantly. In contrast, men with patter 4 or 5 prostate cancer are at increased risk for disease progression, metastasis and death (7, 8, 10). Based on this dichotomy in clinical outcomes between low and high grade prostate cancer, we and other groups have employed RNA based expression profiling and other methods to determine the molecular underpinnings of and develop new markers for high grade prostate cancer.

This invention capitalizes on both our evolved clinical understanding of prostate cancer and recent molecular information regarding higher grade (Gleason pattern 4 or 5) disease in order to identify clinically significant prostate cancer. Namely, we developed a gene list whose differential expression specifically identifies the presence of Gleason pattern 4 or 5 (Gleason sum 7-10) disease. Moreover, we have developed this gene list based on genes encoding for proteins which are secreted, thus allowing the development of assays for clinically significant prostate cancer based on blood and urine.

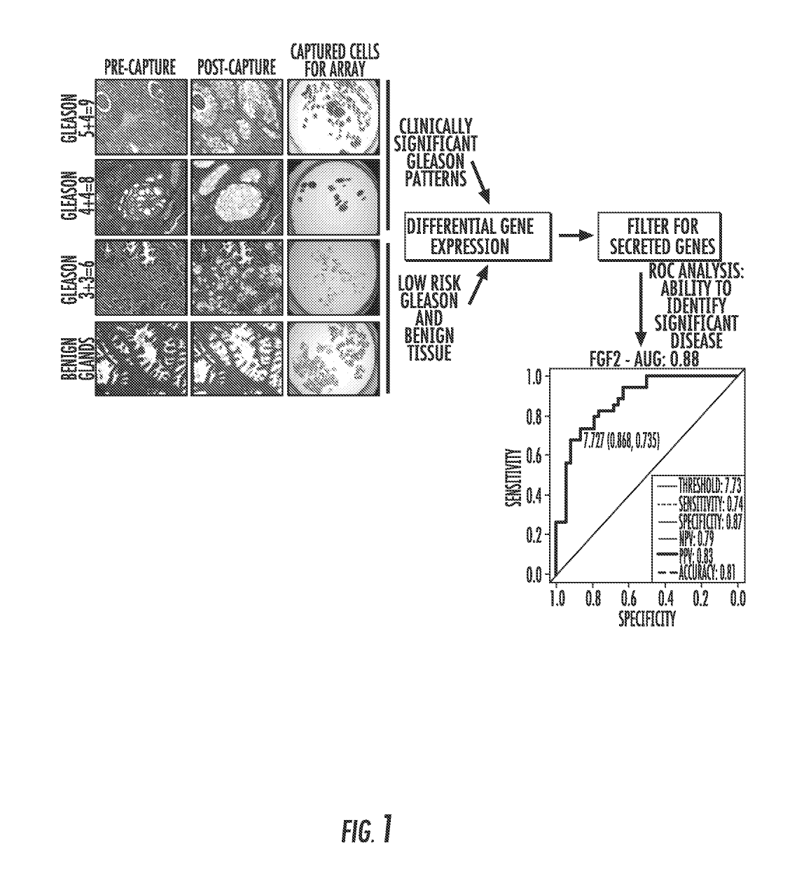

In order to identify markers which could accurately distinguish high grade (clinically significant) prostate cancer, we first performed a series of genome wide expression analyses on prostatectomy tissue from low and high grade prostate cancer as well as tissue form men without prostate cancer and from those with Benign Prostatic Hyperplasia. These studies were performed essentially as described in Ross et al. 2011, and shown schematically in FIG. 1. Genes differentially expressed between Gleason pattern 4 or 5 containing prostate cancer and other tissue types (low grade disease and benign tissue) were then filtered for genes expressing proteins likely to be in the extracellular space by Gene Ontology. A similar process was performed using publically available expression data from an independent cohort of patients from MSKCCC, again comparing Gleason pattern 4 and 5 containing disease to that of low grade cancer and benign tissue. Genes with concordant differential expression in both sets and an area under the curve of at least 0.7 in receiver operating curve analyses were chosen and define the invention. These genes encoding secreted products specific for high grade, clinically significant prostate cancer are listed in Table 1.

TABLE 1
Twenty Gene Set of Secreted Products Distinguishing High Gleason Grade Disease

| | | JHMI Cohort: Gleason Pattern 4 or 5 | | | | | | MSKCCC Cohort: Gleason Pattern 4 or 5 | | | | | |
|---|---|---|---|---|---|---|---|---|---|---|---|---|---|
| | | Disease vs. Gleason Pattern 3, Benign | | | | | | Disease vs. Gleason Pattern 3, Benign | | | | | |
| Gene Symbol | Gene Name | AUC | Sens | Spec | NPV | PPV | Acc | AUC | Sens | Spec | NPV | PPV | AUC |
| FGF2 | fibroblast growth | 0.88 | 0.74 | 0.87 | 0.79 | 0.83 | 0.81 | 0.83 | 0.74 | 0.80 | 0.86 | 0.64 | 0.78 |
| | factor 2 | | | | | | | | | | | | |
| PTGDS | prostaglandin D2 | 0.87 | 0.71 | 0.92 | 0.78 | 0.89 | 0.82 | 0.85 | 0.76 | 0.84 | 0.88 | 0.70 | 0.82 |
| | synthase 21 kDa | | | | | | | | | | | | |
| GPX3 | glutathione | 0.86 | 0.76 | 0.87 | 0.80 | 0.84 | 0.82 | 0.77 | 0.74 | 0.74 | 0.85 | 0.58 | 0.74 |
| | peroxidase 3 | | | | | | | | | | | | |
| PTN | pleiotrophin | 0.86 | 0.76 | 0.84 | 0.80 | 0.81 | 0.81 | 0.87 | 0.68 | 0.96 | 0.86 | 0.88 | 0.87 |
| SERPINF1 | serpin peptidase | 0.85 | 0.88 | 0.71 | 0.87 | 0.73 | 0.79 | 0.87 | 0.82 | 0.86 | 0.91 | 0.74 | 0.85 |
| | inhibitor, clade F | | | | | | | | | | | | |
| | member 1 | | | | | | | | | | | | |
| ANGPTL2 | angiopoietin-like 2 | 0.84 | 0.59 | 1.00 | 0.73 | 1.00 | 0.81 | 0.71 | 0.44 | 0.90 | 0.77 | 0.68 | 0.75 |
| SGCB | sarcoglycan, beta | 0.83 | 0.76 | 0.79 | 0.79 | 0.76 | 0.78 | 0.82 | 0.65 | 0.91 | 0.84 | 0.79 | 0.83 |
| LTBP4 | latent transforming | 0.82 | 0.74 | 0.87 | 0.79 | 0.83 | 0.81 | 0.78 | 0.76 | 0.76 | 0.87 | 0.60 | 0.76 |
| | growth factor beta | | | | | | | | | | | | |
| | binding protein 4 | | | | | | | | | | | | |
| DST | dystonin | 0.81 | 0.91 | 0.63 | 0.89 | 0.69 | 0.76 | 0.80 | 0.65 | 0.90 | 0.84 | 0.76 | 0.82 |
| MMP2 | matrix | 0.80 | 0.62 | 0.92 | 0.73 | 0.88 | 0.78 | 0.79 | 0.68 | 0.86 | 0.85 | 0.70 | 0.80 |
| | metallopeptidase 2 | | | | | | | | | | | | |
| SRGN | serglycin | 0.79 | 0.71 | 0.84 | 0.76 | 0.80 | 0.78 | 0.73 | 0.59 | 0.90 | 0.82 | 0.74 | 0.80 |
| DMD | dystrophin | 0.79 | 0.79 | 0.76 | 0.81 | 0.75 | 0.78 | 0.87 | 0.82 | 0.84 | 0.91 | 0.72 | 0.84 |
| FBLN1 | fibulin 1 | 0.78 | 0.74 | 0.74 | 0.76 | 0.71 | 0.74 | 0.88 | 0.76 | 0.93 | 0.89 | 0.84 | 0.88 |
| ERAP1 | endoplasmic | 0.77 | 0.71 | 0.82 | 0.76 | 0.77 | 0.76 | 0.80 | 0.59 | 0.90 | 0.82 | 0.74 | 0.80 |
| | reticulum | | | | | | | | | | | | |
| | aminopeptidase 1 | | | | | | | | | | | | |
| FXYD6 | FXYD domain | 0.77 | 0.85 | 0.61 | 0.82 | 0.66 | 0.72 | 0.82 | 0.79 | 0.73 | 0.88 | 0.59 | 0.75 |
| | containing ion | | | | | | | | | | | | |
| | transport regulator 6 | | | | | | | | | | | | |
| LGALS3BP | lectin, galactoside- | 0.76 | 0.62 | 0.89 | 0.72 | 0.84 | 0.76 | 0.72 | 0.56 | 0.87 | 0.80 | 0.68 | 0.77 |
| | binding, soluble, 3 | | | | | | | | | | | | |
| | binding protein | | | | | | | | | | | | |
| ANG | angiogenin, | 0.76 | 0.74 | 0.71 | 0.75 | 0.69 | 0.72 | 0.78 | 0.65 | 0.93 | 0.84 | 0.81 | 0.84 |
| | ribonuclease, | | | | | | | | | | | | |
| | RNase A family, 5 | | | | | | | | | | | | |
| GSN | gelsolin | 0.75 | 0.74 | 0.71 | 0.75 | 0.69 | 0.72 | 0.83 | 0.74 | 0.84 | 0.87 | 0.69 | 0.81 |
| SST | somatostatin | 0.75 | 0.62 | 0.89 | 0.72 | 0.84 | 0.76 | 0.74 | 0.56 | 0.86 | 0.80 | 0.66 | 0.76 |
| DKK3 | dickkopf 3 homolog | 0.73 | 0.85 | 0.58 | 0.81 | 0.64 | 0.71 | 0.74 | 0.68 | 0.80 | 0.84 | 0.62 | 0.76 |

1. A method for treating a subject having high grade prostate cancer comprising the steps of:
a. obtaining a biological sample from the subject;
b. performing an assay on the sample obtained from the subject to measure the levels of one or more protein biomarkers listed in Table 1;
c. identifying the subject as having high grade prostate cancer based on a comparison of the measured levels of one or more protein biomarkers to one or more reference controls; and
d. treating the subject with one or more treatment modalities appropriate for a subject having high grade prostate cancer.

2. The method of claim 1, wherein the assay of step (b) comprises contacting the biological sample with one or more capture agents that bind one or more protein biomarkers listed in Table 1 to form a capture agent:protein biomarker complex; and detecting/quantifying the capture agent:protein biomarker complexes.

3. The method of claim 2, wherein the one or more capture agents are antibodies that specifically bind to one or more protein biomarkers listed in Table 1.

4. The method of claim 1, wherein the assay of step (b) is an enzyme linked immunosorbent assay (ELISA).

5. The method of claim 1, wherein the one or more treatment modalities is prostatectomy, radiation therapy, hormone therapy or chemotherapy.

6. The method of claim 1, wherein high grade prostate cancer comprises a Gleason score of 7 or higher.

7. The method of claim 1, wherein the one or more reference control levels comprise a high grade prostate cancer-positive reference control and/or a low grade prostate cancer-positive reference control.

8. A method for identifying a subject as having high grade prostate cancer comprising the steps of:
a. obtaining a biological sample from the subject;
b. performing an assay on the sample obtained from the subject to measure the levels of one or more of FGF2, PTGDS, GPX3, PTN, SERPINF1, ANGPTL2, SGCB, LTBP4, DST, MMP2, SRGN, DMD, FBLN1, ERAP1, FXYD6, LGALS3BP, ANG, GSN, SST, and DKK3; and
c. identifying the subject as having high grade prostate cancer based on a comparison of the measured levels of one or more protein biomarkers to one or more reference controls.

9. The method of claim 8, wherein the assay of step (b) comprises contacting the biological sample with one or more capture agents that bind one or more protein biomarkers listed in Table 1 to form a capture agent:protein biomarker complex; and detecting/quantifying the capture agent:protein biomarker complexes.

10. The method of claim 9, wherein the one or more capture agents are antibodies that specifically bind to one or more protein biomarkers listed in Table 1.

11. The method of claim 8, wherein the assay of step (b) is an enzyme linked immunosorbent assay (ELISA).

12. The method of claim 8, wherein the one or more reference control levels comprise a high grade prostate cancer-positive reference control and/or a low grade prostate cancer-positive reference control.

13. The method of claim 1, wherein the biological sample comprises blood, plasma, serum, urine or stool.

14. A method comprising the step of administering one or more treatment modalities appropriate for a subject having high grade prostate cancer to a subject classified as having high grade prostate cancer based on a comparison of protein expression data of a biomarker panel generated from a biological sample obtained from the subject to a control or reference, wherein the biomarker panel comprises one or more of FGF2, PTGDS, GPX3, PTN, SERPINF1, ANGPTL2, SGCB, LTBP4, DST, MMP2, SRGN, DMD, FBLN1, ERAP1, FXYD6, LGALS3BP, ANG, GSN, SST, and DKK3.

15. The method of claim 14, wherein the one or more treatment modalities is prostatectomy, radiation therapy, hormone therapy or chemotherapy.

16. A method comprises the steps of (a) ordering a diagnostic test that assays protein expression from a biological sample obtained from a patient and classifies the patient as having high grade prostate cancer based on a comparison of protein expression data of a biomarker panel generated from a biological sample obtained from the subject to a control or reference, wherein the biomarker panel comprises one or more of FGF2, PTGDS, GPX3, PTN, SERPINF1, ANGPTL2, SGCB, LTBP4, DST, MMP2, SRGN, DMD, FBLN1, ERAP1, FXYD6, LGALS3BP, ANG, GSN, SST, and DKK3; and (b) administering or prescribing one or more treatment modalities appropriate for a subject having high grade prostate cancer.
